SEQUENCE LISTING (27 sequences)

SEQ ID NO: 1 - protein, 164 aa, Homo sapiens
  pre-pro-AVP
MPDTMLPACFLGLLAFSSACYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 2 - protein, 145 aa, Homo sapiens
  pro-AVP
CYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 3 - protein, 39 aa, Homo sapiens
  CT-pre-proAVP or copeptin
ASDRSNATQLDGPAGALLLRLVQLAGAPEPFEPAQPDAY

SEQ ID NO: 4 - protein, 93 aa, Homo sapiens
  Neurophysin II
AMSDLELRQCLPCGPGGKGRCFGPSICCADELGCFVGTAEALRCQEENYLPSPCQSGQKACGSGGRCAAFGVCCNDESCVTEPECREGFHRRA

SEQ ID NO: 5 - protein, 288 aa, Homo sapiens
  cTnT (Isoform-6)
MSDIEEVVEEYEEEEQEEAAVEEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKQAQTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 6 - protein, 210 aa, Homo sapiens
  cTnI
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 7 - protein, 161 aa, Homo sapiens
  TnC
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVE

SEQ ID NO: 8 - protein, 134 aa, Homo sapiens
  pre-pro-BNP
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 9 - protein, 108 aa, Homo sapiens
  pro-BNP
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 10 - protein, 76 aa, Homo sapiens
  NT-pro-BNP
HPLGSPGSASDLETSGLQEQRNHLQGKLSELQVEQTSLEPLQESPRPTGVWKSREVATEGIRGHRKMVLYTLRAPR

SEQ ID NO: 11 - protein, 32 aa, Homo sapiens
  BNP
SPKMVQGSGCFGRKMDRISSSSGLGCKVLRRH

SEQ ID NO: 12 - protein, 185 aa, Homo sapiens
  pre-pro-ADM
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 13 - protein, 164 aa, Homo sapiens
  pro-ADM
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 14 - protein, 48 aa, Homo sapiens
  MR-pro-ADM
ELRMSSSYPTGLADVKAGPAQTLIRPQDMKGASRSPEDSSPDAARIRV

SEQ ID NO: 15 - protein, 212 aa, Homo sapiens
  pre-pro-ET-1
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 16 - protein, 195 aa, Homo sapiens
  pro-ET-1
APETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 17 - protein, 21 aa, Homo sapiens
  ET-1
CSCSSLMDKECVYFCHLDIIW

SEQ ID NO: 18 - protein, 45 aa, Homo sapiens
  CT-pro-ET-1
RSSEEHLRQTRSETMRNSVKSSFHDPKLKGKPSRERYVTHNRAHW

SEQ ID NO: 19 - protein, 38 aa, Homo sapiens
  Big-ET-1
CSCSSLMDKECVYFCHLDIIWVNTPEHVVPYGLGSPRS

SEQ ID NO: 20 - protein, 116 aa, Homo sapiens
  PCT
APFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 21 - protein, 153 aa, Homo sapiens [sequence omitted]

SEQ ID NO: 22 - protein, 126 aa, Homo sapiens
  pro-ANP
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 23 - protein, 98 aa, Homo sapiens
  NT-proANP
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPR

SEQ ID NO: 24 - protein, 38 aa, Homo sapiens
  MR-proANP
PEVPPWTGEVSPAQRDGGALGRGPWDSSDRSALLKSKL

SEQ ID NO: 25 - protein, 154 aa, Homo sapiens
  Myoglobin
MGLSDGEWQLVLNVWGKVEADIPGHGQEVLIRLFKGHPETLEKFDKFKHLKSEDEMKASEDLKKHGATVLTALGGILKKKGHHEAEIKPLAQSHATKHKIPVKYLEFISECIIQVLQSKHPGDFGADAQGAMNKALELFRKDMASNYKELGFQG

SEQ ID NO: 26 - protein, 381 aa, Homo sapiens
  Creatine kinase
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 27 - protein, 224 aa, Homo sapiens
  C-reactive protein
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

(19) United States
(12) Patent Application Publication
(10) Pub. No.: US 2022/0196678 A1
(43) Pub. Date: Jun. 23, 2022

(54) METHOD FOR THE DIAGNOSIS OF MACCE IN PATIENTS WHO UNDERWENT GASTROINTESTINAL SURGERY

(71) Applicants: B.R.A.H.M.S. GmbH, Hennigsdorf (DE); Linkoping University Hospital, Linkoping (SE)

(72) Inventors: Darius Wilson, Lorrach (DE); Michelle S. Chew, Linkoping (SE); Henrik Andersson, Linkoping (SE)

(21) Appl. No.: 17/432,521

(22) PCT Filed: Feb. 21, 2020
(86) PCT No.: PCT/EP2020/054620

§ 371 Date: Aug. 20, 2021

(30) Foreign Application Priority Data
Feb. 21, 2019 (EP) 19020082.4
Feb. 21, 2019 (EP) 19158645.2

(51) Int. Cl. G01N 33/68

(52) U.S. Cl. CPC G01N 33/6893; G01N 2333/58; G01N 2800/50; G01N 2800/26; G01N 2800/32

(57) ABSTRACT

The invention pertains to a method for the prognosis and/or risk assessment and/or diagnosis of a major adverse cardio- or cerebrovascular event (MACCE) in a patient who underwent gastrointestinal surgery, the method comprising the steps of: i) providing a sample of a bodily fluid from said patient, ii) determining in said sample the level of a biomarker selected from the group consisting of copeptin, troponin and brain natriuretic peptide (BNP), iii) determining at least one additional parameter of said patient, iv) combining the biomarker level determined in step ii) and the additional parameter determined in step iii) into a combined assessment, and v) correlating the combined assessment to said at least one of prognosis, risk assessment and diagnosis of a MACCE in said patient. The invention further pertains to a kit for carrying out the method, a computer and a computer program product comprising computer-executable code being configured to carry out steps iv) and/or v) of the method of the invention.

The present invention pertains to a method for the prognosis and diagnosis of a major adverse cardio- or cerebrovascular event (MACCE) in a patient who has undergone major gastrointestinal surgery. The invention also pertains to a kit for carrying out said method.

Worldwide, more than 200 million adults aged 45 or older undergo major noncardiac surgery each year [Alonso-Coello et al., Polskie Archiwum Medycyny Wewnetrznej. 118(11), pp. 616-618]. Out of these, millions die from complications within 30 days [Botto et al., Anesthesiology, 120(3), pp. 564-578. doi: 10.1097/ALN.0000000000000113] and a significant proportion of patients suffer from major cardiac complications during the first year after surgery [Ekeloef et al., British Journal of Anaesthesia, 117(5), pp. 559-568. doi: 10.1093/bja/aew321]. The prime cause of postoperative mortality is cardiac complications due to ischemic injury of the myocardium, causing cell damage and uncontrolled cell death [Botto et al., Anesthesiology, 120(3), pp. 564-578. doi: 10.1097/ALN.0000000000000113; Sessler and Khanna; Intensive Care Med (2018) 44:811-822, https://doi.org/10.1007/s00134-018-5224-7; Landesberg et al., Circulation, 119(22), pp. 2936-2944. doi: 10.1161/CIRCULATIONAHA.108.828228]. In addition to the number of deaths, postoperative cardiac injuries result in a substantial number of postoperative complications prolonging recovery and hospitalisation, which results in increased patient suffering and medical costs [Devereaux and Sessler, New England journal of medicine, p. 2258, 2015].

MACCE comprise the most common causes of serious perioperative morbidity and mortality and de-pending on the population studied the incidence ranges between 1-7%. 10% of patients who develop MACCE die during their hospital stay. Several independent preoperative and intraoperative risk factors for MACCE have been identified, e.g. coronary artery disease, chronic congestive heart failure, intraoperative hypotension, and blood transfusion. It has been suggested that these are potentially able to stratify patients according to their risk of MACCE. Current risk stratification systems however have only limited sensitivity and specificity. Thus, further research is needed to identify better prediction models and risk stratification scores [Sabaté et al., British Journal of Anaesthesia, p. 879, 2011].

Perioperative myocardial infarction (PMI) is a common cardiovascular complication and is associated with poor outcomes. 12% of patients with PMI die within 30 days and most deaths occur within 48 hours [Hanson et al., Catheterization and Cardiovascular Interventions, 82(4), pp. 622-628. doi: 10.1002/ccd.24626]. However, 58.2% of patients with ischemic myocardial injury do not fulfill the universal definition for myocardial infarction (MI) even though these cardiac injuries are independently associated with 30-day mortality [Botto et al., Anesthesiology, 120(3), pp. 564-578. doi: 10.1097/ALN.0000000000000113; Noordzij et al., British Journal of Anaesthesia, 114(6), pp. 909-918. doi: 10.1093/bja/aev027, 2015].

The third universal definition of MI is necrosis of the myocardium caused by prolonged ischemia and is a common cause of death and disability. This definition was set by the European Society of Cardiology, American College of Cardiology Foundation, American Heart Association, and World Heart Federation. Two distinct mechanisms may cause MI, both of which are seen after noncardiac surgery. A type I MI is caused by an acute coronary syndrome (ACS); a rupture, ulceration, erosion or dissection of an unstable atherosclerotic plaque in one or multiple coronary vessels with subsequent intraluminal thrombosis leading to reduced myocardial blood flow. A type II MI is caused by a prolonged imbalance between myocardial oxygen supply and demand and may arise as a consequence of conditions, such as cardiac arrhythmia, anemia, respiratory failure, hypotension, hypertension or by direct toxic effects by high levels of endogenous or exogenous circulating cortisol and catecholamines [Landesberg et al., Circulation, 119(22), pp. 2936-2944. doi: 10.1161/CIRCULATIONAHA.108.828228; Thygesen et al., Circulation, 126(16), p. 2020. doi: 10.1161/CIR.0b013e31826e1058].

The diagnostic criteria of MI consist of a new elevation of the cardiac biomarker troponin, greater than 3 times the upper level of the reference range in the setting of suspected myocardial ischemia with either an ischemic symptom or an ischemic electrocardiographic (ECG) finding. The necrosis of myocardial cells due to ischemia requires at least 2-4 hours depending on individual variation in e.g. collateral circulation to the ischemic zone, the sensitivity of the myocytes to ischemia, prior myocardial necrosis and coronary arterial size and occlusion. These variations may also impact ECG manifestation of ischemia and therefore an ECG should always be compared to a prior one when available. Acute or evolving ECG abnormalities can potentially allow the clinician to identify the infarction location and the amount of myocardium at risk. The earliest manifestations of myocardial ischemia are typically changes in T-wave and ST-segment. More profound shifts or inversion involving multiple leads/myocardial areas is associated with a greater degree of myocardial ischemia and a worse prognosis. An ECG by itself is often insufficient to diagnose myocardial ischemia and infarction since the ischemic patterns may be caused by other conditions, e.g. ST deviation in acute pericarditis, left bundle branch block (LBBB), left ventricular hypertrophy (LVH) and abnormal Q-waves in e.g. amyloidosis of the heart, acute cor pulmonale and hyperkalemia [Thygesen et al., Circulation, 126(16), p. 2020. doi: 10.1161/CIR.0b013e31826e1058].

The ischemic symptoms of MI include various combinations of nonspecific symptoms, such as chest-, upper extremity-, mandibular pain and dyspnea. Symptoms often last >20 minutes, are diffuse, not localized, positional or affected by movement. MI-patients can also be asymptomatic, this is most common in women, the elderly, diabetics, in post-operative and critically ill patients [Thygesen et al., Circulation, 126(16), p. 2020. doi: 10.1161/CIR.0b013e31826e1058]. Most postoperative myocardial injuries occur within 48 hours after noncardiac surgery. A possible explanation for the lack of symptoms during this period is that the patients usually receive analgesic medications that mask the symptoms. This is thought to partly explain why 65% of the PMI are asymptomatic [Devereaux and Sessler, New England journal of medicine, p. 2258, 2015; Devereaux et al., JAMA, 317(16), pp. 1642-1651. doi: 10.1001/jama.2017.4360, 2017].

Myocardial injury after noncardiac surgery (MINS), is a prognostically relevant myocardial injury due to ischemia occurring in 8% of surgical patients [Botto et al., Anesthesiology, 120(3), pp. 564-578. doi: 10.1097/ALN.0000000000000113]. 10% of patients suffering from MINS die within 30 days, MINS-patients also have a greater 1-year mortality as well as beyond 1 year and a greater incidence of nonlethal cardiac arrest, congestive heart failure and stroke [Botto et al., Anesthesiology, 120(3), pp. 564-578. doi: 10.1097/ALN.0000000000000113; Mauermann et al., Current Opinion in Anaesthesiology, 29(3), pp. 403-412. doi: 10.1097/ACO.0000000000000336]. Furthermore, the regional myocardial injury can stun the myocardium, resulting in diastolic dysfunction and reduced cardiac output resulting in reduced peripheral blood supply, impaired healing of tissue and increased risk of wound infection [Noordzij et al., British Journal of Anaesthesia, 114(6), pp. 909-918. doi: 10.1093/bja/aev027, 2015].

The diagnostic criteria for MINS is a postoperative troponin elevation judged to be due to myocardial ischemia (no evidence of nonischemic etiology) with or without ischemic symptoms. MINS does not include perioperative myocardial damage originating from another etiology than ischemia, e.g. sepsis or pulmonary embolism. The postoperative thresholds for MINS depend on the type of troponin assay. For 5^{th} generation cTnT, the threshold is >20 ng/L with an increase from baseline of at least 5 ng/L or >65 ng/L [Sessler and Khanna; Intensive Care Med (2018) 44:811-822, https://doi.org/10.1007/s00134-018-5224-7]. The criteria for MINS cover a wider spectrum of myocardial injury than MI, from reversible myocardial injury to necrosis. This broader concept comprises several manifestations of myocardial injury and makes MINS a more applicable term for surgical patients and lowers the risk of underestimating the extent of myocardial damage seen after noncardiac surgery [Mauermann et al., Current Opinion in Anaesthesiology, 29(3), pp. 403-412. doi: 10.1097/ACO.0000000000000336].

93.6% of MINS occur within 3 days of surgery, MINS patients will have mild troponin elevation and often lack ischemic features [Noordzij et al., British Journal of Anaesthesia, 114(6), pp. 909-918. doi: 10.1093/bja/aev027, 2015]. 84% of patients suffering from MINS do not experience ischemic symptoms and only 34.9% will show signs of ischemia on ECG [Botto et al., Anesthesiology, 120(3), pp. 564-578. doi: 10.1097/ALN.0000000000000113]. These findings indicate that a reliable diagnosis of perioperative myocardial injury, in most cases, is only possible by continuous postoperative troponin measurements. Otherwise, it is reported that 93.1% of MINS and 68% of PMI may go undetected [Devereaux et al., JAMA, 317(16), pp. 1642-1651. doi: 10.1001/jama.2017.4360, 2017].

The mechanism of MINS remains unclear, but is suggested to be similar to the mechanisms behind PMI [Mauermann et al., Current Opinion in Anaesthesiology, 29(3), pp. 403-412. doi: 10.1097/ACO.0000000000000336]. Several trials have been conducted trying to counteract MINS, but there are still no known methods safely and efficiently preventing the myocardial injuries [Sessler and Khanna; Intensive Care Med (2018) 44:811-822, https://doi.org/10.1007/s00134-018-5224-7]. One example, the POISE-study, a large randomized trial administering beta blockers perioperative and postoperative, managed to reduce the occurrence of major cardiovascular events by 30% but at the same time increased the incidence of serious hypotension, stroke and mortality [Devereaux et al., Lancet, 371(9627), pp. 1839-1847. doi: 10.1016/S0140-6736(08)60601-7].

Additionally, the development of an infection after surgery constitutes one of the major postoperative complications and causes pain, poor wound healing, a need for further treatment including antibiotics, longer hospital stays and increased health care costs. Postoperative infections may cause severe problems including failure of the surgical procedure, sepsis, organ failure and in some cases ultimately death. A postoperative infection may be defined as any infection that occurs within 30 days of operation and may be related to the operation itself or the postoperative course. The incidence of postoperative infections can vary widely and depends on the type of procedure performed and the patient's inherent risk factors for the development of infection. Postoperative infections can for example involve the wound itself or deeper infections within body cavities or systemic infections, but may also include more distant infections such as pulmonary infection (e.g. pneumonia) or catheter associated urinary infections.

Postoperative wound infections, also known as surgical site infections (SSIs), complicate the recovery course of many patients. As defined by the Centers for Disease Control and Prevention (CDC), these infections typically occur within 30 days of an operation at the site or part of the body where the surgery took place. SS's may be classified as superficial/incisional if limited to the skin and subcutaneous tissue, as deep incisional when involving the fascia and muscle, or as organ space when involving a body cavity (e.g. in the abdominal cavity following gastrointestinal surgery; Horan et al., Am J Infect Control; 36(5):309-332, 2008). Deep tissue and organ space SS's are less frequently encountered than superficial SSIs, but are associated with greater morbidity/mortality, readmission rates, longer hospital stay and increased overall hospital-associated costs when compared with superficial SSIs. Although the majority of SS's are uncomplicated, others may be severe and more challenging, often requiring extensive surgical debridement, multiple reoperations and may be life-threatening.

Postoperative infections may arise to a number of factors in addition to an infection at the surgical site. Post-operative pneumonia, and respiratory tract infection are for instance generally regarded as a manifestation of physiological and immune compromise, which occurs after major surgery. Postoperative pulmonary infections are associated with cough, phlegm, shortness of breath, chest pain, temperature above 38° C., and pulse rate above 100 a minute. Up to half of people may have asymptomatic chest signs after surgery, and up to a quarter develop symptomatic disease. The incidence of postoperative pulmonary complications in major surgery ranges from <1% to 23%. Several studies have shown pulmonary complications to be more common than cardiac complications, and postoperative respiratory failure is a common complication postoperatively (Miskovic at al., British Journal of Anaesthesia, 118 (3): 317-34, 2017).

Diagnosing a surgical site infection can be challenging, as the signs and symptoms are frequently nonspecific and may lack sensitivity depending on the clinical scenario. However, an early diagnosis of postsurgical infection is of critical importance in order to initiate appropriate therapy such as antibiotic treatment.

A particularly severe postsurgical infection relates to a blood infection or sepsis. Despite significant improvements in diagnostic and preventative measures, the incidence of sepsis has continued to escalate rapidly in hospitalized patients (Martin et al. 2003), with mortality rates ranging between 10% and 54%, depending on the level of disease severity, definition of organ dysfunction used, and country specific incidence (Kaukonen et al. 2014, Vincent et al. 2006). For any postoperative disease, but in particular for sepsis, an early and accurate assessment of both the infectious load and disease severity, in terms of the overall pathophysiological host response, is of crucial importance in order to make prompt and reliable decisions concerning diagnostic testing and treatment strategies.

Previous studies investigated the use of biomarkers in predicting postoperative outcomes in patients. For example, a correlation of the expression of arginine vasopressin (AVP) and copeptin to the severity of systemic inflammatory response syndrome (SIRS) with and without shock in patients after non-cardiac surgery could be shown [Jochberger et al., SHOCK, Vol. 31, No. 2, pp. 132-138, 2009]. Copeptin rises even higher in SIRS patients after cardiac surgery [Jochberger et al., J Clin Endocrinol Metab 91:4381-4386, 2006]. Another study showed that copeptin is increased in patients with inflammatory bowel disease (IBD) and even more increased in IBD patients who underwent bowel resections [Ohlsson and Melander, Drug Target Insights 2015:9, 21-27 DOI: 10.4137/DTI.S26589]. Copeptin as a general stress biomarker is known to rise in response to surgical trauma, however, no difference between minimally invasive and conventional surgical techniques could be found [Netto et al., Acta Chirurgica Belgica, DOI: 10.1080/00015458.2018.1482698, 2018]. Pre-operative copeptin and NT-proBNP levels have been investigated to improve risk stratification for patients undergoing major vascular surgery, showing that increased biomarker levels are predictive of outcome in this patient group [Jarai et al., Am J Cardiol 2011; 108:1188-1195, 2011]. However, another study showed that the predictive quality of copeptin for major adverse cardiac events was lost when evaluating patients undergoing vascular surgery while in severe stages of chronic kidney disease (CKD) [Schrimpf et al., PLoS ONE 10(4):e0123093. DOI: 10.1371/joumal.pone.0123093, 2015]. In summary, while there have been indications about possible predictive value of biomarkers for the outcome of postoperative patient's health, there is no clear method allowing an objective assessment of a patient's situation. There is therefore a need for methods allowing a quick and early assessment of possible MACCE and MINS as well as postsurgical infection in patients undergoing abdominal surgery, specifically gastrointestinal surgery, to reduce postoperative complications prolonging recovery and hospitalisation and increasing mortality.

A primary objective of the present invention therefore resides in the provision of a method and kit which allow a quick and reliable diagnosis, prognosis and rule-out of MACCE and/or MINS and/or postsurgical infection in patients who undergo major gastrointestinal surgery. Further objects will become apparent from the following description.

The objective is reached by a method and a kit according to the independent claims. Preferred embodiments are described in the respective dependent claims.

Specifically, the invention relates to a method for at least one of prognosis, risk assessment and diagnosis of a major adverse cardio- or cerebrovascular event (MACCE) in a patient who has undergone major gastrointestinal surgery, the method comprising the steps of:
i) providing a sample of a bodily fluid from said patient,
ii) determining in said sample the level of a biomarker selected from the group consisting of copeptin, troponin and brain natriuretic peptide (BNP),
iii) determining at least one additional parameter of said patient,
iv) combining the biomarker level determined in step ii) and the additional parameter determined in step
iii) into a combined assessment, and
v) correlating the combined assessment to said at least one of prognosis, risk assessment and diagnosis of a MACCE in said patient.

Especially, the invention relates to a method for the diagnosis of a major adverse cardio- or cerebrovascular event (MACCE) in a patient who underwent gastrointestinal surgery, wherein step v) relates to correlating the combined assessment to the presence or absence of a MACCE in said patient. It is stressed that the invention relates to an in-vitro method. The invention is not practiced on a human or animal body, but separated from these bodies and completely in-vitro. Thus, the step of providing the patient sample is not meant to be practiced upon human or animal body either, but for example relates to providing an in-vitro assay with samples, for example, by taking samples from a refrigerator. The actual sample extraction from human or animal body is not part of the inventive method.

The inventors realized that the specific biomarkers copeptin, troponin and BNP allow a quick and early diagnosis of whether or not a patient who underwent gastrointestinal surgery has MACCE. Moreover the inventors realized that the specific biomarkers were able to provide important prognostic information by identifying patients at risk of MACCE prior to surgery. The combination of the level of at least one of these biomarkers with at least one additional parameter shows improved results so that the resulting assessment outperforms those of each of the biomarkers alone. While the invention pertains to the field of clinical diagnostics, the determination of the biomarker levels is carried out on samples taken from the patient and is thus not practiced on the body of the patient.

In a preferred embodiment, the method is a method for at least one of prognosis, risk assessment and diagnosis of a major adverse cardio- or cerebrovascular event (MACCE) in combination with an infection in a patient who underwent gastrointestinal surgery, preferably a method for risk stratification of said patients, stratifying said patients into a group more likely to develop both MACCE and an infection and another group less likely to develop both MACCE and an infection. It is entirely surprising that, based upon the measurement of the biomarkers described herein, an accurate and reliable conclusion can be made as to whether the patient postsurgically suffers a cardiovascular or cerebrovascular event, such as MINS or MACCE, and an infection, such as a blood infection. This prognostic ability of copeptin, troponin and/or BNP in the particular settings described herein is novel and surprising, and enables improvements in the detection, prediction and management of adverse events surrounding operations. The combined assessment of these possible adverse events is also of particularly high clinical relevance. The optimal therapy guidance may crucially depend on such an assessment since measures or medication for treating an infection may be unfavorable for the cardiovascular or cerebrovascular event or vice versa. By providing a diagnosis or prognosis for both adverse events the clinical personnel may choose the best possible therapeutic approach. When predicting or diagnosing e.g. a MACCE, PMI or a MINS the subsequent clinical procedures, such as a bypass surgery or an angioplasty, elevate the risk of the deterioration of a possible infection. An early preemptive anti-infective therapy, such as an antibiotic therapy, may be of critical importance for an overall positive patient outcome.

As used herein, “infection” within the scope of the invention means a pathological process caused by the invasion of normally sterile tissue or fluid by pathogenic or potentially pathogenic agents/pathogens, organisms and/or microorganisms, and relates preferably to infection(s) by bacteria, viruses, fungi, and/or parasites. Accordingly, the infection can be a bacterial infection, viral infection, and/or fungal infection. The infection can be a local or systemic infection. For the purposes of the invention, a viral infection may be considered as infection by a microorganism.

Further, the subject suffering from an infection can suffer from more than one source(s) of infection simultaneously. For example, the subject suffering from an infection can suffer from a bacterial infection and viral infection; from a viral infection and fungal infection; from a bacterial and fungal infection, and from a bacterial infection, fungal infection and viral infection, or suffer from a mixed infection comprising one or more of the infections listed herein, including potentially a superinfection, for example one or more bacterial infections in addition to one or more viral infections and/or one or more fungal infections.

As used herein “infectious disease” comprises all diseases or disorders that are associated with bacterial and/or viral and/or fungal infections.

In one embodiment the infection to be detected or to be tested for may be selected from species of Bordetella, such as Bordetella pertussis, Borrelia, such as Borrelia burgdorferi, Brucella, such as Brucella abortus, Brucella canis, Brucella melitensis or Brucella suis, Campylobacter, such as Campylobacter jejuni, Chlamydia and Chlamydophila, such as Chlamydia pneumonia, Chlamydia trachomatis, Chlamydophila psittaci, Clostridium, such as Clostridium botulinum, Clostridium difficile, Clostridium perfringens, Clostridium tetani, Corynebacterium, such as Corynebacterium diphtheria, Enterococcus, such as Enterococcus faecalis, Enterococcus faecium, Escherichia, such as Escherichia coli, Francisella, such as Francisella tularensis, Haemophilus, such as Haemophilus influenza, Helicobacter, such as Helicobacter pylori, Legionella, such as Legionella pneumophila, Leptospira, such as Leptospira interrogans, Listeria, such as Listeria monocytogenes, Mycobacterium, such as Mycobacterium leprae, Mycobacterium tuberculosis, Mycobacterium ulcerans, Mycoplasma, such as Mycoplasma pneumonia, Neisseria, such as Neisseria gonorrhoeae, Neisseria meningitides, Pseudomonas, such as Pseudomonas aeruginosa, Rickettsia, such as Rickettsia rickettsia, Salmonella, such as Salmonella typhi, Salmonella typhimurium, Shigella, such as Shigella sonnei, Staphylococcus, such as Staphylococcus aureus, Staphylococcus epidermidis, Staphylococcus saprophyticus, Streptococcus, such as Streptococcus agalactiae, Streptococcus pneumonia, Streptococcus pyogenes, Treponema, such as Treponema pallidum, Vibrio, such as Vibrio cholera, Yersinia, such as Yersinia pestis, Yersinia enterocolitica or Yersinia pseudotuberculosis.

Pathogenic fungi are fungi that cause disease in humans or other organisms. Candida species are important human pathogens that are best known for causing opportunist infections in immunocompromised hosts (e.g. transplant patients, AIDS patients, cancer patients). Infections are difficult to treat and can be very serious: 30-40% of systemic infections result in death. Aspergillosis is another potential fungal pathogen. Aspergillus can cause disease in three major ways: through the production of mycotoxins; through induction of allergenic responses; and through localized or systemic infections. With the latter two categories, the immune status of the host is pivotal. The most common pathogenic species are Aspergillus fumigatus and Aspergillus flavus. Aspergillus flavus produces aflatoxin which is both a toxin and a carcinogen and which can potentially contaminate foods. Aspergillus fumigatus and Aspergillus clavatus can cause disease. Cryptococcus neoformans can cause disease in humans. Cryptococcus neoformans is the major human and animal pathogen. Cryptococcus laurentii and Cryptococcus albidus have been known to occasionally cause moderate-to-severe disease in human patients with compromised immunity. Cryptococcus gattii is endemic to tropical parts of the continent of Africa and Australia and can cause disease. Histoplasma capsulatum can cause histoplasmosis in humans, dogs and cats. Pneumocystis jirovecii (or Pneumocystis carinii) can cause a form of pneumonia in people with weakened immune systems, such as premature children, the elderly, and AIDS patients. Stachybotrys chartarum or “black mould” can cause respiratory damage and severe headaches.

In one embodiment the infection to be detected or to be tested for may be selected from Acinetobacter baumannii, Klebsiella pneumoniae, Acinetobacter lwoffii, Listeria monocytogenes, Aeromonas caviae, Morganella morganii, Aeromonas hydrophila, Neisseria gonorrhoeae, Aspergillus flavus, Neisseria meningitidis, Aspergillus nidulans, Pasteurella multocida, Aspergillus niger, Pasteurella pneumotropica, Aspergillus terreus, Propionibacterium acnes, Bacillus anthracis, Proteus mirabillis, Bacillus cereus, Providencia rettgeri, Bacillus subtilis, Pseudomonas aeruginosa, Bacteroides fragilis, Salmonella choleraesuis, Brucella melitensis, Serratia liquefaciens, Burkholderia cepacia, Serratia marcescens, Candida albicans, Staphylococcus aureus, Candida dubliniensis, Staphylococcus epidermidis, Candida glabrata, Staphylococcus haemolyticus, Candida krusei, Staphylococcus hominis, Candida parapsilosis, Staphylococcus saccharolyticus, Candida tropicalis, Staphylococcus warneri, Capnocytophaga canimorsus, Stenotrophomonas maltophilia, Citrobacter braakii, Streptococcus agalactiae, Citrobacter freundii, Streptococcus anginosus, Clostridium perfringens, Streptococcus bovis, Corynebacterium jeikeium, Streptococcus constellatus, Enterobacter aerogenes, Streptococcus dysgalactiae, Enterobacter cloacae, Streptococcus mutans, Enterobacter sakazakii, Streptococcus pneumoniae, Enterococcus faecalis, Streptococcus pyogenes, Enterococcus faecium, Streptococcus salivarius, Escherichia coli, Streptococcus sanguinis, Shigella sp., Streptococcus suis, Gemella haemolysans, Vibrio vulnificus, Gemella morbillorum, Yersinia enterocolitica, Haemophilus influenzae, Yersinia pestis, Kingella kingae, Yersinia pseudotuberculosis and; Klebsiella oxytoca.

As used herein the term “blood infection” may comprise a systemic blood stream infection, a sepsis, severe sepsis and/or septic shock.

“Sepsis” in the context of the invention refers to a systemic response to infection. Alternatively, sepsis may be seen as the combination of SIRS with a confirmed infectious process or an infection. Sepsis may be characterized as clinical syndrome defined by the presence of both infection and a systemic inflammatory response (Levy et al., Crit Care Med.; 31(4):1250-6, 2003). The term “sepsis” used herein includes, but is not limited to, sepsis, severe sepsis, septic shock.

The term “sepsis” used herein includes, but is not limited to, sepsis, severe sepsis, septic shock. Severe sepsis refers to sepsis associated with organ dysfunction, hypoperfusion abnormality, or sepsis-induced hypotension. Hypoperfusion abnormalities include lactic acidosis, oliguria and acute alteration of mental status. Sepsis-induced hypotension is defined by the presence of a systolic blood pressure of less than about 90 mm Hg or its reduction by about 40 mm Hg or more from baseline in the absence of other causes for hypotension (e.g. cardiogenic shock). Septic shock is defined as severe sepsis with sepsis-induced hypotension persisting despite adequate fluid resuscitation, along with the presence of hypoperfusion abnormalities or organ dysfunction (Bone et al., CHEST 101(6): 1644-55, 1992).

The term sepsis may alternatively be defined as life-threatening organ dysfunction caused by a dysregulated host response to infection. For clinical operationalization, organ dysfunction can preferably be represented by an increase in the Sequential Organ Failure Assessment (SOFA) score of 2 points or more, which is associated with an in-hospital mortality greater than 10%. Septic shock may be defined as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dl) in the absence of hypovolemia.

The term “sepsis” used herein relates to all possible stages in the development of sepsis.

The term “sepsis” also includes severe sepsis or septic shock based on the SEPSIS-2 definition (Bone et al., 2009). The term “sepsis” also includes subjects falling within the SEPSIS-3 definition (Singer et al., JAMA;315(8):801-810, 2016). The term “sepsis” used herein relates to all possible stages in the development of sepsis.

The at least one additional parameter can be selected from the group consisting of the level of at least one additional biomarker and a clinical parameter of said patient. When using more than one additional parameter in the combined assessment, the additional parameters can be of the same category or different categories. In case of two additional parameters, for example, the additional parameters can both be a level of an additional biomarker, two clinical parameters or the level of an additional biomarker plus a clinical parameter. The additional biomarker level can be the level of one of the two remaining biomarkers mentioned in step ii) not used for determining the biomarker level in said step. If, for example, the biomarker level determined in step ii) is that of copeptin, the additional parameter may be the level of troponin or BNP.

More generally speaking, determining at least one additional parameter preferably comprises determining the level of at least one additional biomarker which is selected from the group consisting of copeptin, tro-ponin, BNP, mid-regional proadrenomedullin (MR-proADM), C-terminal proendothelin-1 (CT-proET-1), procalci-tonin (PCT), MR-proANP (mid-regional pro atrial natriuretic peptide), creatinine kinase, creatine kinase-MB, myoglobin, lactate and CRP (C-reactive protein).

In a preferred embodiment, the levels of all three biomarkers copeptin, troponin and brain natriuretic peptide (BNP) are determined and used in the following steps iv) and v). Optionally, at least one of MR-proADM, CT-proET-1 and PCT can additionally be determined and combined into the combined assessment.

The sequence of the 164 amino acid precursor peptide of Vasopressin (pre-pro-Vasopressin) is given in SEQ ID NO:1. Pro-Vasopressin relates to the amino acid residues 19 to 164 of the sequence of pre-pro-Vasopressin. The amino acid sequence of pro-Vasopressin is given in SEQ ID NO:2. Pro-Vasopressin is cleaved into mature Vasopressin, Neurophysin II and C-terminal proVasopressin (CT-proAVP or copeptin). Copeptin relates to amino acid residues 126 to 164 of pre-pro-Vasopressin. The amino acid sequence of copeptin is provided in SEQ ID NO:3. Neurophysin II comprises the amino acid residues 32 to 124 of pre-pro-Vasopressin and its sequence is shown in SEQ ID NO:4.

Troponin is a protein found in muscles which facilitate contraction by sliding of actin and myosin filaments. It comprises three subunits, C, I and T. An isoform of troponin T, cTnT, is only found in cardiomyocytes and is the most important cardiac biomarker because of its high myocardial specificity and clinical sensitivity. However, troponin levels typically show a delay in their increase after surgery, so that their usefulness for early diagnosis of MACCE and MINS in post-operative patients has typically been regarded as limited. The sequence of the subunit C is given in SEQ ID NO:7. The sequence of the subunit I is given in SEQ ID NO:6. The sequence of the subunit T is given in SEQ ID NO:5.

The sequence of the 134 amino acid precursor peptide of brain natriuretic peptide (pre-pro-BNP) is given in SEQ ID NO:8. Pro-BNP relates to amino acid residues 27 to 134 of pre-pro-BNP. The sequence of proBNP is shown in SEQ ID NO:9. Pro-BNP is cleaved into N-terminal pro-BNP (NT-pro-BNP) and mature BNP. NT-pro-BNP comprises the amino acid residues 27 to 102 and its sequence is shown in SEQ ID NO:10. The SEQ ID NO:11 shows the sequence of BNP comprising the amino acid residues 103 to 134 of the pre-pro-BNP peptide.

A clinical parameter in the sense of this invention generally means any parameter indicative of the patient's health and/or physical condition. The clinical parameter is preferably selected from a parameter which is significant of a cardio- or cerebrovascular condition or event. Preferably, the clinical parameter is selected from the group consisting of age, abnormal ECG (especially pathological Q-waves), LBBB (development of left bundle branch block), ST-elevation (2 mm in V1, V2, V3 or 1 mm in other leads), ST-depression (development of ST-segment depression 1 mm), T-wave inversion (symmetric inversion of T-waves 1 mm in at least two continuous leads), intraoperative hypotension, intraoperative tachycardia, intraoperative bradycardia, hyperlipidaemia, smoking status, anaemia, functional capacities (METs), red-blood cell transfusion, arrhythmias, rhythm other than sinus, duration of the gastrointestinal surgery and operation size, patient history (especially in regard of cardiac and cerebrovascular events) and renal disease. The invention also comprises any of the parameters as mentioned herein alone or in any combination with any number of the other parameters.

As there is no distinct, explicit and exclusive definition of MACCE and which conditions this term includes in the art, MACCE as used herein is defined as the occurrence of at least one condition selected from the following group of conditions:
Non-fatal cardiac arrest (absence of cardiac rhythm or presence of chaotic rhythm requiring any component of basic or advanced cardiac life support),
acute myocardial infarction (increase and gradual decrease in troponin level or a faster increase and decrease if creatine kinase isoenzyme as marker of myocardial necrosis in the company of at least one of the following: Ischemic symptoms, abnormal Q-waves on the ECG, ST-segment elevation or depression, coronary artery intervention (e.g. coronary angioplasty) or an atypical decrease in an elevated troponin level detected at its peak after surgery in a patient without documented alternative explanation for the tro-ponin elevation),
congestive heart failure (new in-hospital signs or symptoms of dyspnea or fatigue, orthopnea, paroxysmal nocturnal dyspnea, increased jugular venous pressure, pulmonary rales on physical examination, cardiomegaly or pulmonary vascular engorgement),
new cardiac arrhythmia (ECG evidence of atrial flutter, atrial fibrillation, or second- or third-degree atrioventricular conduction block),
angina (dull diffuse substernal chest discomfort precipitated by exertion or emotion and relieved by rest or glyceryl trinitrate),
stroke (embolic, thrombotic, or hemorrhagic event lasting at least 30 min with or without persistent residual motor, sensory, or cognitive dysfunction; if the neurological symptoms continue for >24 h, a person is diagnosed with stroke, and if lasting <24 h the event is defined as a transient ischemic attack),
transient ischemic attack (embolic, thrombotic, or hemorrhagic event lasting at least 30 min with or without persistent residual motor, sensory, or cognitive dysfunction; if the neurological symptoms continue for >24 h, a person is diagnosed with stroke, and if lasting <24 h the event is defined as a transient ischemic attack) and
myocardial injury after noncardiac surgery (MINS; postoperative troponin elevation judged to be due to myocardial ischemia (no evidence of nonischemic etiology) with or without ischemic symptoms).

Other conditions not mentioned above, for example, circulatory shock, are therefore not considered a MACCE as used herein and are explicitly excluded.

“Diagnosis” in the context of the present invention relates to the recognition and early detection of a disease or clinical condition or its absence in a subject and may also comprise differential diagnosis and rule outs. Early in this regard is intended to mean the earliest possible moment for a diagnosis. This includes patients with and without disease related symptoms. The diagnosis is realized by the measurement of biomarkers with or without the addition of further clinical parameters, scores or medicinal procedures known in the art. Again, the present invention relates to an in-vitro diagnosis. The diagnosis is practiced upon samples of a patient, but not on a human or animal body.

In the present invention, the term “risk assessment” denotes an assignment of a probability to experience certain adverse events to an individual. Hereby, the individual may preferably be accounted to a certain risk category, wherein categories comprise for instance high risk versus low risk, or risk categories based on numerical values, such as risk category 1, 2, 3, etc. In the present invention, the term “risk stratification” relates to the grouping of subjects into different risk groups according to their further prognosis. Risk stratification also relates to stratification for applying preventive and/or therapeutic measures.

In the present invention, the term “prognosis” denotes a prediction of how a subject's (e.g. a patient's) medical condition will progress. This may include an estimation of the chance of recovery or the chance of an adverse outcome for said subject (e.g. occurrence of a MACCE with or without an infection or death).

The term “sample” as used herein refers to a sample of bodily fluid obtained for the purpose of diagnosis, prognosis, or evaluation of a subject of interest, such as a patient. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

Thus, in a preferred embodiment of the invention the sample is selected from the group comprising a blood sample, a serum sample, a plasma sample, a cerebrospinal fluid sample, a saliva sample and a urine sample or an extract of any of the aforementioned samples. Preferably, the sample is a blood sample, most preferably a serum sample or a plasma sample.

The term “level” in the context of the present invention relates to the concentration (preferably expressed as weight/volume or molar amount/volume) of marker peptides taken from a sample of a patient.

The term “patient” as used herein refers to a living human or non-human organism that is receiving medical care or that should receive medical care due to a disease. This includes persons with no defined illness who are being investigated for signs of pathology, specifically persons who are investigated for a possible MACCE or MINS, preferably in combination with infection. Thus the methods and assays described herein are applicable to both, human and veterinary disease. Specifically, the patients evaluated in the inventive method undergo, preferably an elective, gastrointestinal surgery. The patient can also suffer from other comorbidities like hypertension, diabetes, COPD (chronic obstructive pulmonary disease), metabolic syndrome, obesity (BMI (body mass index) of at least 30), non-cardiovascular chronic disease, pain, fever, infection, sepsis or SIRS (systemic inflammatory response syndrome).

The term “combining” as used herein for the provision of a combined assessment, refers to the assignment of an indicative value to a measured or calculated result and the combination of values from different results into the combined assessment. It may comprise selection of a certain number of parameters and the arrangement of these parameters into specified groups using a mathematical algorithm (e.g. deviation or ratio). For example, a measured biomarker level can lie above or below a predetermined threshold, which the skilled person can translate into an indicative value by evaluating whether or not an increase in the biomarker value in comparison to reference data is considered indicative of the presence of a disease or condition. Different thresholds may provide different indicative strengths. The same applies to calculated results, e.g. decrease or increase ratios, ratios between different biomarker levels and so on. The term “combined assessment” therefore relates to the indicative value of the entirety of all measured or calculated results as provided by the present invention, which can then be correlated to a condition in a patient.

The term “correlating”, as used herein in reference to the combined assessment, refers to comparing the entirety of the indicative value of all measured or calculated results (the combined assessment) to their value in persons known to suffer from, or known to be at risk of, a given condition. An assessment in a patient sample can be compared to a value known to be associated with a specific diagnosis. The sample's assessment is said to have been correlated with a diagnosis; that is, the skilled artisan can use the assessment to determine whether the patient suffers from a specific type of a disease, and respond accordingly. Alternatively, the assessment can be compared to an assessment known to be associated with a good outcome (e.g. the absence of disease etc.). In preferred embodiments, a panel of marker levels and their combined indicative value is correlated to a global probability or a particular outcome.

In the present invention, all of the patients the inventive method is practiced upon undergo a gastrointestinal surgery. The aim of the inventive method is to determine whether or not these patients have or are at risk of a MACCE, preferably a MACCE in combination with an infection. Therefore, the correlation of step iv) of the inventive method preferably includes providing reference data pertaining to patients who underwent gastrointestinal surgery and who did and/or did not have a MACCE or MINS, preferably a MACCE or MINS in combination with an infection. The combined assessment of the patient is then compared to this reference data to determine whether or not the patient has or is at risk of a MACCE or a MINS, preferably a MACCE or MINS in combination with an infection. A reference group consisting of patients who also underwent gastrointestinal surgery is better suited for the present invention than, for example, a reference group of healthy patients.

To increase the performance of the inventive method further, in a preferred embodiment, the level of at least one additional biomarker in the sample is determined, the additional biomarker being selected from the group consisting of copeptin, troponin, BNP, mid-regional proadrenomedullin (MR-proADM), C-terminal proendothelin-1 (CT-proET-1), procalcitonin (PCT), MR-proANP (mid-regional pro atrial natriuretic peptide), creatinine kinase, creatine kinase-MB, myoglobin, lactate and CRP (C-reactive protein). The level of the at least one additional biomarker is combined into the combined assessment. In an especially preferred embodiment, the levels of all of the biomarkers copeptin, troponin, BNP, MR-proADM, CT-proET-1 and PCT are determined and combined into the combined assessment. Employing a combination of biomarkers increases the diagnostic performance of the present method and makes the method less susceptible to fluctuations of the single biomarkers.

The sequence of the 153 amino acid pre-proANP is shown in SEQ ID NO:21. Upon cleavage of an N-terminal signal peptide (25 amino acids) and the two C-terminal amino acids, proANP (SEQ ID NO:22) is released. “Pro-atrial natriuretic peptide” or “proANP” refers to the pro-hormone comprising 126 amino acids. As used herein, a peptide comprising 28 amino acids (99-126) of the C-terminal section of a pro-hormone comprising 126 amino acids (proANP) is referred to as the actual hormone ANP. Upon release of ANP from its pro-hormone proANP, an equimolar amount of the remaining larger partial peptide of proANP, the N-terminal proANP, consisting of 98 amino acids (NT-proANP; proANP (1-98); shown as SEQ ID NO:23) is released into circulation. As NT-proANP possesses a significantly greater half life time and stability NT-proANP can be used as laboratory parameter for diagnosis, follow-up and therapy control; see, for example, Lothar Thomas (Editor), Labor and Diagnose, 5th expanded ed., sub-chapter 2.14 of chapter 2, Kardiale Diagnostik, pages 116-118, and WO 2008/135571. The level of proANP is preferably measured in the plasma or serum of a subject.

“Atrial natriuretic peptide (ANP)”, a member of the natriuretic peptide family, regulates several physiological parameters including diuresis and natriuresis, and lower arterial blood pressure (BP). It is predominantly produced in the atrium of the heart and comprises 98% of natriuretic peptides in the circulation (Vesely D L. Life 2002; 53:153-159). ANP is derived from the cleavage of its precursor pro-hormone, which is significantly more stable in the circulation than the mature peptide. A midregional fragment of the precursor hormone (amino acids 53-90 of NT-proANP; SEQ ID NO:24), called midregional-proANP (MR-proANP), may be relatively resistant to degradation by exoproteases, unlike epitopes in the N- or C-terminals of proANP used in previous immunoassays (Morgenthaler N G et al. Clin Chem 2004; 50:234-236; Gerszten R E et al. 2008. Am J Physiol Lung Cell Mol Physiol). We have previously noted that in adults with essential hypertension, higher plasma levels of MR-proANP were associated with higher systolic blood pressure (SBP), greater arterial stiffness and severity of hypertension (unpublished data). Based on these findings, we hypothesized that plasma MR-proANP may be associated with measures of target organ damage in adults with hypertension.

As used herein, “lactate” refers to the lactate concentration measured in the blood. Normally, the lactate concentration is assessed daily or even more often. The lactate concentration in the blood can be determined by lactate oxidase spectrophotometric methods. As for clinical applications, it is well known in the art that lactate can identify patients with an increased morbidity and mortality risk (Broder G, Weil M H. Excess lactate: An index of reversibility of shock in human patients. Science 1964; 143: 1457-59; Manikis P, Jankowski S, Zhang H, Kahn R J, Vincent J L. Correlation of serial blood lactate levels to organ failure and mortality after trauma. Am J Emerg Med 1995; 13, 6: 619-22.)

“Myoglobin” is a single-chain globular protein of 153 amino acids (SEQ ID NO:26), containing a heme (iron-containing porphyrin) prosthetic group in the center around which the remaining apoprotein folds. Its sequence is shown in SEQ ID NO:25. Myoglobin is a sensitive marker for muscle injury, making it a potential marker for heart attack in patients with chest pain (Weber et al. 2005. Clinical Biochemistry 38: 1027-30). CK-MB and cardial troponin T cTnT are used in combination with ECG, and the clinical signs to diagnose Acute Myocardial Infarction (AMI). Cardial troponin is a protein complex consisting of the three subunits T (cTcT), I (cTcI) and C, of which T and I are only located in heat muscle tissue and are used as markers for diagnostic purposes (Rottbauer W et al., Eur Heart J 1996; 17:3-8).

“Creatine kinase” is an enzyme and is represented by different isoforms that could have a B-(brain type) or M- (muscle type) subunit. Creatine kinase comprises the isoforms CK-MM, CK-MB and CK-BB, that can have distinct expression pattern based on the tissue type (Schlattner U, Tokarska-Schlattner M, Wallimann T (February 2006). “Mitochondrial creatine kinase in human health and disease”. Biochimica et Biophysica Acta. 1762 (2): 164-80.) The enzyme plays a functional role in the adenosine triphosphate (ATP) metabolism and is therefore present in organs such as muscle, retina, brain, heart or kidney that have a high demand for ATP. Blood levels of creatine kinase are detected to detect tissue damage in associated diseases as for example myocardial infarction, muscular dystrophy or acute kidney injury. (Wallimann T, Wyss M, Brdiczka D, Nicolay K, Eppenberger H M (January 1992). “Intracellular compartmentation, structure and function of creatine kinase isoenzymes in tissues with high and fluctuating energy demands: the ‘phosphocreatine circuit’ for cellular energy homeostasis”. The Biochemical Journal. 281 (Pt 1) (1): 21-40; Moghadam-Kia S, Oddis C V, Aggarwal R (January 2016). “Approach to asymptomatic creatine kinase elevation”. Cleveland Clinic Journal of Medicine. 83 (1): 37-42).

“C-reactive protein (CRP)” is an acute-phase protein, which is physiologically induced during inflammatory conditions (Thompson D, Pepys M B, Wood S P (1999). “The physiological structure of human C-reactive protein and its complex with phosphocholine”. Structure. 7 (2): 169-77). The sequence of CRP is given in SEQ ID NO:27. CRP is part of the complement system by promoting phagocytosis of Macrophages. Moreover, CRP can also activate the innate immune response against infections (Bray C, Bell L N, Liang H, Haykal R, Kaiksow F, Mazza J J, Yale S H (December 2016). “Erythrocyte Sedimentation Rate and C-reactive Protein Measurements and Their Relevance in Clinical Medicine”). Wisconsin Medical Journal (WMJ). 115 (6): 317-21.). CRP is known and widely used as a marker for inflammation. In healthy adults, the normal concentrations of CRP varies between 0.8 mg/L to 3.0 mg/L and in rare occasions up to 10 mg/L. Pathological levels can be defined as follows: mild inflammation and viral infections (10-40 mg/L), active inflammation, bacterial infection (40-200 mg/L), severe bacterial infections and burns (>200 mg/L) (Chew K S (April 2012). “What's new in Emergencies Trauma and Shock? C-reactive protein as a potential clinical biomarker for influenza infection: More questions than answers”. Journal of Emergencies, Trauma, and Shock. 5 (2): 115-7.).

The amino acid sequence of the precursor peptide of Adrenomedullin (pre-pro-Adrenomedullin) is given in SEQ ID NO:12. Pro-Adrenomedullin relates to amino acid residues 22 to 185 of the sequence of pre-pro-Adrenomedullin. The amino acid sequence of pro-Adrenomedullin (proADM) is given in SEQ ID NO:13. MR-pro-Adrenomedullin (MR-proADM) relates to amino acid residues 45-92 of pre-proADM. The amino acid sequence of MR-proADM is provided in SEQ ID NO:14.

The sequence of the 212 amino acid precursor peptide of Endothelin-1 (pre-pro-Endothelin-1) is given in SEQ ID NO:15. Pro-ET-1 relates to the amino acid residues 18 to 212 of the sequence of pre-pro-ET-1. The amino acid sequence of pro-ET-1 is given in SEQ ID NO:16. Pro-ET-1 is cleaved into mature ET-1, big-ET-1 and C-terminal proET-1 (CT-proET-1). ET-1 relates to the amino acid residues 53 to 73 of pre-pro-ET-1. The amino acid sequence of ET-1 is shown in SEQ ID NO:17. CT-proET-1 relates to amino acid residues 168 to 212 of pre-pro-ET-1. The amino acid sequence of CT-proET-1 is provided in SEQ ID NO:18. Big-ET-1 comprises the amino acid residues 53 to 90 of pre-pro-ET-1 and its sequence is shown in SEQ ID NO:19.

Procalcitonin is a precursor of calcitonin and katacalcin. The amino acid sequence of PCT 1-116 is given in SEQ ID NO:20.

Generally, the levels of the mentioned biomarkers can be measured by directly determining the biomarkers themselves. However, in a preferred embodiment, the level of at least one of the biomarkers is determined by determining the level of at least one of a precursor, a precursor fragment and a fragment of the biomarker. In other words, the levels of the biomarkers are determined indirectly by determining the levels of precursors, precursor fragments or fragments of the biomarkers. The advantage lies in the fact that these indirect peptide targets are often more easily measured or are more stable either in the patient's bodily fluid or in the assay environment. As the ratios of the indirect peptide targets to the biomarkers of interest are known, the levels of the indirect peptide targets can easily be used to determine the levels of the biomarkers of interest.

As mentioned herein in the context of proteins and other peptides, the term “fragment” refers to smaller proteins or peptides derivable from larger proteins or peptides, which hence comprise a partial sequence of the larger protein or peptide. Said fragments are derivable from the larger proteins or peptides by saponification of one or more of its peptide bonds. “Fragments” of the biomarkers preferably relate to fragments of at least 6 amino acids in length, most preferably at least 12 amino acid residues in length. Such fragments are preferably detectable with immunological assays.

For example, in a preferred embodiment, determining the level of troponin comprises determining the level of the subunit cardiac troponin T (cTnT), preferably isoform 6 of cTnT (SEQ ID NO:5) or a homologous peptide with at least 75% amino acid sequence identity with isoform 6 of cTnT. More preferably, the amino acid sequence identity with isoform 6 of cTnT is at least 80%, at least 85%, at least 90% and, most preferably, at least 95%. Cardiac troponin T is released from the myocardium through tissue damage and is therefore used as a biomarker for MI. There are several isoforms of cTnT and the current invention is not restricted to either one of them, while isoform 6 is preferred. However, any of the other isoforms can also be used in the present invention.

In another preferred embodiment, determining the level of BNP comprises determining the level of precursor fragment NT-proBNP (SEQ ID NO:10).

As the risk for postoperative complications increases with patient age, it is preferred that the inventive method is practiced on patients who are at least 50 years old. Limiting the patient population on which the method is practiced enables cut-off values to be specifically chosen for the biomarker levels prevalent in the target population. By evaluating patients with at least 50 years of age, the inventive method can be tailored to the physiological parameters of this group.

The present invention is directed to the diagnoses of serious complications following gastrointestinal surgery. Therefore, as severe complications are more predominant after severe surgical procedures, it is preferred that the patient spent at least one day in a hospital after undergoing the gastrointestinal surgery. The length of the hospital stay reflects the severity of the surgical procedure and therefore the general risk of complications. The invention can also be practiced on a sample of a patient being treated with analgesics or pain treatment. By directing the method to this group of patients, the inventive diagnosis is directed to the ones who potentially are most in need.

Eligible gastrointestinal surgery procedures can be either laparoscopic or open (conventional). Specifically, the gastrointestinal surgery is selected from the group consisting of
stomach surgery,
small intestine surgery, in particular duodenum surgery,
large intestine surgery, in particular rectum surgery,
reproductive system surgery, in particular hysterectomy or salpingoophorectomy,
kidney surgery, in particular nephrectomy,
urinary bladder surgery, in particular cystectomy,
gallbladder surgery, in particular cholecystectomy, and
surgery of gastrointestinal cysts.

As the present method is directed to the diagnosis of MACCE, preferably combined with infection, resulting from gastrointestinal surgery, it is preferred that the patient is free from cardiovascular comorbidities. Such comorbidities often also lead to nonphysiological levels of the biomarkers as described herein and could thus obscure or be mistaken as newly acquired MACCE as a result of the gastrointestinal surgery. The diagnostic value of the present method is therefore higher in patients without pre-existing conditions that could be mistaken as MACCE.

Accordingly, in a preferred embodiment, the patient is excluded if an exclusion condition is present in the patient, the exclusion condition being selected from the group consisting of organ transplantation, traumatic injury, endocrine disease, endocrine surgery, vascular disease, vascular surgery (e.g. surgery of an aneurism), endovascular disease, endovascular surgery, acute coronary syndrome (ACS), heart failure, decompensated congestive heart failure, aortic stenosis, reduced left ventricular ejection fraction (LVEF), and circulatory shock. The exclusion condition can either lie or have been diagnosed in the recent past of the patient or be diagnosed shortly after the gastrointestinal surgery. The main criterion is that the exclusion condition present in the patient influences the levels of the biomarkers according to the present method at the time of sampling of the patient. If this is the case and these circumstances are timely identified, the patient is excluded. In this way, the detrimental effect of the exclusion condition on the diagnostic performance of the present invention can be prevented. Otherwise, the present method might lead to the wrong diagnosis of a MACCE in a patient under the influence of one or more of the exclusion conditions.

As the present invention aims at a quick diagnosis of a MACCE, preferably combined with an infection, in the aftermath of gastrointestinal surgery, the sample is preferably taken at a time shortly before and/or after the gastrointestinal surgery or within the first three days after the surgery. Specifically, it is preferred that the sample has been taken from the patient no more than 24 hours before the gastrointestinal surgery, no more than 24 hours after the gastrointestinal surgery, on day one after the gastrointestinal surgery, on day two after the gastrointestinal surgery or on day three after the gastrointestinal surgery. While taking the sample after the gastrointestinal surgery is of course suitable to monitor the effects of the gastrointestinal surgery itself on the level of the biomarkers, taking the sample shortly before the gastrointestinal surgery has two main applications. First of all, it serves to establish a baseline of the levels of the biomarkers as described herein in the specific patient. The levels of the biomarkers taken at later points in time therefore can be compared to these baselines. Second, higher levels of the biomarkers as mentioned herein in comparison to a healthy control group even in patients without one of the previously mentioned exclusion conditions can signify an increased risk of this specific patient for developing a MACCE, preferably combined with an infection, after the gastrointestinal surgery. Therefore, the measurement of the levels of the biomarkers prior to the gastrointestinal surgery can serve to increase the diagnostic value of the inventive method. The control group can also be a group of patients who underwent gastrointestinal surgery and did or preferably did not develop a MACCE or MINS, preferably combined with an infection.

The inventive method yields valuable results even when only one sample of the patient taken at one of the previously mentioned points in time is evaluated. However, the performance of the inventive method is increased when multiple samples of the patient taken at different points in time are evaluated. In a preferred embodiment, the inventive method therefore comprises providing of from two to five samples from the patient, wherein the samples are taken at different times before and/or after the gastrointestinal surgery, and wherein steps ii), iv) and v) are practiced on all said samples. In the most preferred embodiment, five samples are taken from the patient at different points in time shortly before and after the gastrointestinal surgery. Preferably, the different times said of from two to five samples are taken are selected from the group consisting of no more than 24 hours before the gastrointestinal surgery, no more than 24 hours after the gastrointestinal surgery, day one after the gastrointestinal surgery, day two after the gastrointestinal surgery and day three after the gastrointestinal surgery. In the embodiment wherein five samples are taken, the respective sample is taken at one of these points in time. The determination of the biomarker levels in these samples can either be done sequentially, for example each sample can be measured on the day it is taken. Alternatively, the samples from all points of time can be collected and measured simultaneously, for example after the last sample has been taken. For different biomarkers, different assays or one multi-marker collective assay can be used.

In the embodiments of the present method in which more than one sample of the patient is provided, the development of the levels of the biomarkers in the samples, for example an increase or decrease in the biomarker levels between the points in time the samples were taken, can be used to increase the diagnostic value of the invention. For example, in a preferred embodiment, the levels of said biomarkers in the samples taken at different times are determined and then compared, and the differences in the levels of the biomarkers at different times are combined into the combined assessment. By comparison of the levels in samples taken at different times, additional information about the condition of the patient can be obtained, going beyond the biomarker levels at a specific time. As already mentioned, this additional information can be an increase or a decrease of a specific biomarker over the period in which samples were taken. However, additional information can also be gained through a more complex evaluation of the relationship of each of the biomarkers to the others. For example, the significance of a high level of copeptin on day one after the gastrointestinal surgery, generally being an indicator for a MACCE, is either increased if the patient also shows a high level of troponin and/or BNP on day two and/or three or decreased if the level of troponin and/or BNP does not show an increase at these times. In this way, the levels of the biomarkers measured in the samples taken at different times develop a synergy in the diagnostic value of the inventive method, going way beyond the mere levels of the biomarkers at one or more points in time (i.e. the information that a biomarker level dropped by a certain value from one sampling time to another has more diagnostic value than the information about the biomarker levels at these sampling times alone).

In a preferred embodiment, a biomarker level determined to be above a specific threshold value is indicative of a MACCE, preferably combined with an infection, in the patient. For the value of this specific threshold, several factors have to be taken into account, as will be explained below.

First of all, the sensitivity and specificity of a diagnostic and/or prognostic test depend on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy; herein specifically patients undergoing gastrointestinal surgery and not developing a MACCE with or without an infection) and “disease” populations (i.e. patients suffering from diabetes, insulin resistance and/or metabolic syndrome; herein specifically patients undergoing gastrointestinal surgery and developing a MACCE and potentially also an infection). For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve (AUC) is a measure of the probability that the measured marker level will allow correct identification of a disease or condition. Thus, the area under the ROC curve can be used to determine the effectiveness of the test. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art (See, e.g., Hanley et al. 1982. Radiology 143: 29-36). Preferably, ROC curves result in an AUC of greater than about 0.5, more preferably greater than about 0.7, still more preferably greater than about 0.8, even more preferably greater than about 0.85, and most preferably greater than about 0.9. The term “about” in this context refers to +/−5% of a given measurement. In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

It is therefore important to note that the specific threshold values used in the inventive method have to be adapted to the use of the invention in practice. Different threshold values will lead to different sensitivity and specificity of the invention, which should be adapted by the clinician using the invention with consideration of the specific situation. Thus, one might adopt the threshold value depending on whether it is considered more appropriate to identify most of the subjects at risk at the expense of also identifying “false positives”, or whether it is considered more appropriate to identify mainly the subjects at high risk at the expense of missing several subjects at moderate risk. These decisions have to be taken by the clinician practicing the present invention and are implemented by a modification of the threshold values. The threshold values as given herein are therefore exemplary and are not to be understood to restrict the invention in any way.

Another point to consider is that different assays may need different threshold values if these assays have been calibrated differently. Therefore the above mentioned threshold values shall apply for such differently calibrated assays accordingly, taking into account the differences in calibration. One possibility of quantifying the difference in calibration is a method comparison analysis (correlation) of the assay in question (e.g. a copeptin assay) with the respective biomarker assay used in the present invention (e.g. Thermo Scientific B⋅R⋅A⋅H⋅MS Kryptor Compact plus) by measuring the respective biomarker (e.g. copeptin) in samples using both methods. Another possibility is to determine with the assay in question, given this test has sufficient analytical sensitivity, the median biomarker level of a representative normal population, compare results with the median biomarker levels as described in the literature and recalculate the calibration based on the difference obtained by this comparison.

All in all, the specific threshold values used in the present invention have to be determined on an individual basis whenever the invention is employed. As exemplary values, and in a preferred embodiment of the invention, the specific threshold value above which a biomarker level is indicative of a MACCE, preferably combined with an infection, in the patient is selected from the group consisting of
25 pmol/L, preferably 75 pmol/L, more preferably 125 pmol/L, even more preferably 175 pmol/L and most preferably 225 pmol/L for copeptin,
15 ng/L, preferably 20 ng/L, more preferably 25 ng/L, even more preferably 30 ng/L and most preferably 35 ng/L, 45 ng/L or 65 ng/L for cTnT,
500 ng/L, preferably 900 ng/L, more preferably 1300 ng/L, even more preferably 1700 ng/L and most preferably 2100 ng/L or 2800 ng/L for NT-proBNP,
0.8 nmol/L or 1.0 nmol/L, preferably 1.25 nmol/L, more preferably 1.5 nmol/L, even more preferably 1.75 nmol/L and most preferably 2.0 nmol/L or 2.4 nmol/L for MR-proADM,
80 pmol/L, preferably 90 pmol/L, more preferably 100 pmol/L, even more preferably 110 pmol/L and most preferably 120 pmol/L for CT-proET-1, and
0.5 μg/L, preferably 1.0 μg/L, more preferably 1.5 μg/L, even more preferably 2.0 μg/L and most preferably 2.5 μg/L or 3.0 μg/L for PCT.

In another specific embodiment, the threshold lies within a margin of plus or minus 10% of any of the abovementioned thresholds. The threshold for copeptin could thus for example also be anywhere between 22.5 pmol/L and 27.5 pmol/L (e.g. in a range of 25 pmol/L plus or minus 10%). This applies to all the exemplary threshold values given herein.

In further specific embodiments, the risk of a MACCE, preferably combined an with infection, in the patient pertains to the risk of a MACCE, preferably combined with an infection, in the patient within at least one of 30 days and 12 months after the gastrointestinal surgery. These time spans have proven to be most important in clinical prognosis.

Apart from the mere occurrence of a MACCE, preferably combined with an infection, the present invention may also be used to assess the risk of death of the patient, specifically death from developing a MACCE, preferably combined with an infection, after gastrointestinal surgery. It is therefore preferred that the prognosis comprises the risk of mortality within at least one of 30 days and 12 months after the gastrointestinal surgery.

The important subgroup of MACCE, myocardial injury after noncardiac surgery (MINS), has already been mentioned. As MINS is responsible for a large number of complications and prolonged hospitalizations including increased patient mortality, in a preferred embodiment of the invention, the MACCE is a MINS and the method is a method for the diagnosis of a MINS, preferably combined with an infection, in a patient who underwent gastrointestinal surgery. In other words, the inventive method can be directed to the specific diagnoses and/or prognosis pertaining to MINS, preferably combined with an infection, in a patient who underwent gastrointestinal surgery. As MINS typically do not sufficiently show any specific symptoms in other clinical tests, including ECG, the invention is of high practical value as patients developing a MINS might otherwise go undetected and therefore untreated.

In a preferred embodiment of the invention, therefore, a biomarker level determined to be above a specific threshold value is indicative of a MINS, preferably combined with an infection, in the patient. The general remarks pertaining to the definition and the determination of specific threshold values as mentioned above of course also apply to this specific threshold values in connection with MINS, preferably combined with an infection.

In another preferred embodiment of the invention, a biomarker level determined to be above a specific threshold value is indicative of a perioperative myocardial injury in the patient. The general remarks pertaining to the definition and the determination of specific threshold values as mentioned above of course also apply to this specific threshold values in connection with perioperative myocardial injury.

As exemplary values, and in a preferred embodiment of the invention, the specific threshold value above which a biomarker level is indicative of a MINS, preferably combined with an infection, in the patient is selected from the group consisting of
25 pmol/L, preferably 50 pmol/L or 75 pmol/L, more preferably 125 pmol/L, even more preferably 175 pmol/L and most preferably 225 pmol/L for copeptin,
10 ng/L, preferably 20 ng/L, more preferably 30 ng/L, even more preferably 40 ng/L and most preferably 50 ng/L or 60 ng/L for cTnT,
250 ng/L or 500 ng/L, preferably 750 ng/L, more preferably 1250 ng/L or 1500 ng/L, even more preferably 1750 ng/L and most preferably 2250 ng/L for NT-proBNP,
0.8 nmol/L or 1.0 nmol/L, preferably 1.25 nmol/L, more preferably 1.5 nmol/L, even more preferably 1.75 nmol/L and most preferably 2.0 nmol/L for MR-proADM,
65 pmol/L or 75 pmol/L or 80 pmol/L, preferably 90 pmol/L, more preferably 100 pmol/L, even more preferably 110 pmol/L and most preferably 120 pmol/L for CT-proET-1, and
0.2 μg/L or 0.5 μg/L, preferably 0.75 μg/L or 0.8 μg/L, more preferably 1.0 μg/L, even more preferably 1.25 μg/L and most preferably 1.5 μg/L for PCT.

In another specific embodiment, the threshold lies within a margin of plus or minus 10% of any of the abovementioned thresholds. The threshold for copeptin could thus for example also be anywhere between 22.5 pmol/L and 27.5 pmol/L (e.g. in a range of 25 pmol/L plus or minus 10%). This applies to all the exemplary threshold values given herein.

One further option when determining the specific threshold values above which the level of the biomarker is indicated of a MACCE and/or MINS, preferably combined with an infection, is to also considering the point in time when the sample in question was taken from the patient. For example, the level of a specific biomarker, for example, copeptin, is elevated shortly after the gastrointestinal surgery both in patients developing a MACCE and/or MINS and in patients who do not develop such complications. This increase, however, quickly drops again. Therefore, when evaluating the levels of copeptin, a higher specific threshold should be chosen for the sample taken at most 24 hours after the gastrointestinal surgery in comparison to any other point in time. This can be done for any of the biomarkers as mentioned herein. It is therefore a preferred embodiment of the present invention, that the determined biomarker levels taken at different times are evaluated using different threshold values. In this way, the physiological baseline of the biomarker levels as determined in patients not developing a MACCE and/or MINS with or without an infection can be considered and taken into account in the inventive method.

Another increase in the diagnostic performance of the invention may be achieved if the determined biomarker levels taken at different times are differently weighted. For example, an increased level of troponin in samples taken on day two and/or on day three after the gastrointestinal surgery is more significant than an increased level of troponin in a sample taken before the gastrointestinal surgery. Therefore, increased levels on day two and/or on day three after the gastrointestinal surgery can be given more diagnostic weight in the combined assessment, meaning that these increased levels at these specific points in time influence the combined assessment more than less significant measurements at other points in time. The different weight assigned to biomarker levels taken at different times can also pertain to different biomarkers. For example, an increased level of tro-ponin in samples taken on day two and/or on day three after the gastrointestinal surgery can be more significant than an increased level of copeptin on day one and/or two and/or three after the gastrointestinal surgery. By weighting the determined biomarker levels according to their diagnostic value at the specific points in time of their respective measurement, the performance of the invention can be increased.

By providing multiple samples from different points in time from the patient, these samples and their significance for the diagnosis and/or prognosis of the patient can be assessed in many different ways. For example, a relative change in the biomarker level between two samples taken at different times can be combined into the combined assessment. A relative change is measured as a decrease or an increase of the level of a specific biomarker expressed in relation to a previous level, for example, as a percentage. It can, for example, be determined by how much the biomarker increased or decreased in view to one or more previous measurements. These measurements do not have to be sequential. For example, the increase or decrease of the level of a biomarker could be determined from the measurement prior of the gastrointestinal surgery to the first and/or the second and/or the third and/or the fourth postoperative measurement. In this way, each of the samples taken at a specific point in time can be compared to any other sample from any other point in time. This can differ for each of the biomarkers. For example, the method could comprise using the relative change of BNP from a sample taken on the second day to the third day after the surgery and additionally the relative change of copeptin from the sample taken at most 24 hours after the gastrointestinal surgery to the sample taken on the first day after the surgery. Any such combinations are possible.

In another preferred embodiment of the present invention, a ratio between the levels of at least two biomarkers in the sample is determined and said ratio is combined into the combined assessment, wherein the biomarkers for determining the ratio are preferably selected from the pairs copeptin/troponin, copeptin/BNP and BNP/troponin. The level of the at least two biomarkers can be from one sample or of different samples, specifically different samples taken at different points in time. The ratio can be calculated between the measured levels of the biomarkers directly or parameters derived from the levels of the biomarkers, for example the relative change of the biomarker levels. In a preferred embodiment, the ratio between the relative changes of at least two biomarkers is determined and combined into the combined assessment. In another preferred embodiment, the ratios determined from samples taken at different times are compared to obtain a relative change of the ratios over time, preferably expressed as percentage, which is then also combined into the combined assessment.

For example, the method also comprises providing reference data for the determined biomarkers and especially for the determined parameters as described above, for example the difference between the biomarker levels at two different points in time, the relative change of the biomarker levels or the ratios of the biomarker levels. The reference data can then be used to compare the measured results and determine the status of the patient. In this preferred embodiment, step iv) comprises providing reference data for the determined biomarkers and at least one value selected from the group consisting of
the determined level of at least one of the biomarkers,
the differences in the level of at least one of the biomarkers taken at different times,
the relative change in the levels of one of said biomarkers determined in two samples taken at different times,
the ratio between the levels of at least two biomarkers either taken at the same time or taken at different times and
the relative change of the ratios of at least two biomarkers determined in at least two samples taken at different times
   is compared to the reference data, wherein the difference of the value as determined and the reference data is computed, and wherein the computed difference is preferably expressed in the form of a score, especially as a numerical value.

Reference data is preferably also provided with regard to a clinical parameter as the additional parameter. The reference data is again preferably provided from a reference group consisting of patients who also underwent gastrointestinal surgery, but reference data from a reference group of healthy patients is principally also suitable. As previously stated, the clinical parameter determined in the patient is compared to the reference data and the result is preferably transferred into a score, particularly a numerical value. While a clinical parameter can be determined at the same time(s) as the biomarker level(s) it will only make sense to take more than one value in case significant changes of the clinical parameter can be expected in the time frame in question. Clinical parameters, such as age, smoking habits, patient history and the like, need obviously only be taken once. Other clinical parameters, such as ECG, may be taken at several points in time. The relative change of the values thus obtained may be evaluated in an analogous way as described for the biomarker levels.

The term “score” in the context of the present invention refers to a rating, especially expressed numerically, based on the specific achievement or the degree to which certain qualities or conditions (e.g. the level of biomarkers or derived parameters) are present in said patient.

It is further preferred that at least two different scores are determined and the combined assessment comprises a combined score determined from said at least two different scores indicative of the presence or absence of a MACCE, preferably combined with an infection, in the patient. In other words, the method comprises a scoring system in which each of the determined parameters, i.e., at least one biomarker level and at least one additional parameter, are expressed as scores, representing intermediate results, which are then combined into a combined score, from which the desired diagnosis and/or prognosis can be derived. For example, the reference data is divided into groups of values, with their values increasing or decreasing, preferably linearly. The increase or decrease is suitably in line with the severity of the outcome of the patients from which the reference data was taken. Each of the groups is associated with a respective score, for example, such that the number increases with severity. The values determined for the patient in accordance with steps ii) and iii) are then compared with the reference values associated with the predefined groups. The score of the appropriate group is then attributed to the determined parameter and used for evaluating the combined score, which then forms the basis for the final assessment. This evaluation is done for each of the parameters determined in steps ii) and iii), that is, the level of at least one biomarker and the at least one additional parameter. In the same way, biomarker levels at specific points in time or their differences or their relative changes or their ratios as described above can be evaluated and expressed as scores which are then used in the combined score.

As these scores and the combined score can be expressed as numerical values, they can also be graded according to the degree of their difference to the reference data. This results in a graded combined score, which may serve to stratify the patient into a specific risk group pertaining to the diagnosis and/or the prognosis of the patient having or developing a MACCE or MINS, preferably combined with an infection. Such risk groups may for example pertain to a low risk, a medium risk or a high risk of having or developing a MACCE or MINS, preferably combined with an infection, specifically in the time frames as mentioned above. The stratification can also pertain to the risk of death, also specifically in the time frames as mentioned above. The score as described can also be used for therapeutic guidance, with different risk groups being indicative of the need of different therapeutic approaches for the specific patient.

The reference data can be data obtained from healthy subjects. These subjects can be free from any known pathology and not undergo gastrointestinal surgery. However, it is preferred that the reference data pertains to patients who underwent gastrointestinal surgery and who did and/or did not have a MACCE or MINS with or without an infection. The comparison of the data obtained from the patient as described above with reference data from patients also undergoing gastrointestinal surgery but not developing a MACCE or MINS with or without an infection is especially meaningful, which is why this is the most preferred embodiment.

In the following, embodiments relating to determining a level of at least one of the biomarkers copeptin, troponin and BNP in combination with the level of at least one of the biomarkers proADM, PCT and proET-1 or fragment(s) thereof for diagnosing or predicting MACCE in combination with an infection will be described.

### ADM/Any Infection:

In one embodiment determining a level of at least one additional biomarker comprises determining the level of proADM or fragment(s) thereof and a level of proADM or fragment(s) thereof above a threshold value indicates an elevated likelihood of an infection.

In one embodiment the sample is isolated preoperatively and determining a level of at least one biomarker comprises determining the level of proADM or fragment(s) thereof and a level of proADM or fragment(s) thereof above a threshold value±20% or more selected from a range of values in between 0.7 nmol/L and 0.8 nmol/L indicates an elevated likelihood of an infection. Any value within this range may be considered as an appropriate threshold value. For example, 0.70, 0.71, 0.72, 0.73, 0.74, 0.75, 0.76, 0.77, 0.78, 0.79, 0.8 nmol/L.

In one embodiment the sample is isolated postsurgically within 12 hours, preferably within 6 hours after surgery and determining a level of at least one biomarker comprises determining the level of proADM or fragment(s) thereof and a level of proADM or fragment(s) thereof above a threshold value±20% or more selected from a range of values in between 1.0 nmol/L and 1.2 nmol/L indicates an elevated likelihood of an infection. Any value within this range may be considered as an appropriate threshold value. For example, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2 nmol/L.

In one embodiment the sample is isolated postsurgically one day after surgery and determining a level of at least one biomarker comprises determining the level of proADM or fragment(s) thereof and a level of proADM or fragment(s) thereof above a threshold value±20% or more selected from a range of values in between 1.3 nmol/L and 1.5 nmol/L indicates an elevated likelihood of an infection. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample is isolated postsurgically two days after surgery and determining a level of at least one biomarker comprises determining the level of proADM or fragment(s) thereof and a level of proADM or fragment(s) thereof above a threshold value±20% or more selected from a range of values in between 1.3 nmol/L and 1.5 nmol/L indicates an elevated likelihood of an infection. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.1 nmol/L and 1.5 nmol/L indicates an elevated likelihood an infection. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample is isolated postsurgically and determining a level of at least one biomarker comprises determining the level of proADM or fragment(s) thereof and a level of proADM or fragment(s) thereof above a threshold value±20% or more selected from a range of values in between 1.1 nmol/L and 1.5 nmol/L indicates an elevated likelihood of an infection. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample is isolated postsurgically at one day, two days, three days or more after surgery and determining a level of at least one biomarker comprises determining the level of proADM or fragment(s) thereof and a level of proADM or fragment(s) thereof above a threshold value±20% or more selected from a range of values in between 1.3 nmol/L and 1.5 nmol/L indicates an elevated likelihood of an infection. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment a first sample is isolated from the patient postsurgically at a first point in time, preferably one day or more after surgery, and a second sample is isolated from the patient postsurgically at a subsequent second point in time, wherein preferably between the first and second point in time at least 12 hours, 24 hours, 36 hours, 48 hours or more have passed and determining a level of at least one biomarker comprises determining the level of proADM or fragment(s) thereof and an increase or a leveling in the level of proADM or fragment(s) thereof indicates an elevated likelihood of an infection.

In one embodiment a first sample is isolated from the patient postsurgically at a first point in time, preferably one day or more after surgery, and a second sample is isolated from the patient postsurgically at a subsequent second point in time, wherein preferably between the first and the second point in time at least 12 hours, 24 hours, 36 hours, 48 hours or more have passed and determining a level of at least one biomarker comprises determining the level of proADM or fragment(s) thereof and an increase of the level of proADM or fragment(s) thereof above a threshold value±20% or more selected from a range of values in between 0.01 nmol/L and 0.4 nmol/L indicates an elevated likelihood of an infection. Any value within this range may be considered as an appropriate threshold value for the increase of proADM or fragment(s) in between the first and second point in time. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4 nmol/L.

### ADM/Blood Infection:

In one embodiment determining a level of at least one additional biomarker comprises determining the level of proADM or fragment(s) thereof and a level of proADM or fragment(s) thereof above a threshold value indicates an elevated likelihood of a blood infection.

In one embodiment the sample is isolated preoperatively and determining a level of at least one biomarker comprises determining the level of proADM or fragment(s) thereof and a level of proADM or fragment(s) thereof above a threshold value±20% or more selected from a range of values in between 0.7 nmol/L and 0.9 nmol/L indicates an elevated likelihood of a blood infection. Any value within this range may be considered as an appropriate threshold value. For example, 0.70, 0.71, 0.72, 0.73, 0.74, 0.75, 0.76, 0.77, 0.78, 0.79, 0.8, 0.81, 0.82, 0.83, 0.84, 0.84, 0.85, 0.86, 0.87, 0.88. 0.89, 0.9 nmol/L.

In one embodiment the sample is isolated postsurgically within 12 hours, preferably within 6 hours after surgery and determining a level of at least one biomarker comprises determining the level of proADM or fragment(s) thereof and a level of proADM or fragment(s) thereof above a threshold value±20% or more selected from a range of values in between 1.0 nmol/L and 1.3 nmol/L indicates an elevated likelihood of a blood infection. Any value within this range may be considered as an appropriate threshold value. For example, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 nmol/L.

In one embodiment the sample is isolated postsurgically one day after surgery and determining a level of at least one biomarker comprises determining the level of proADM or fragment(s) thereof and a level of proADM or fragment(s) thereof above a threshold value±20% or more selected from a range of values in between 1.2 nmol/L and 1.8 nmol/L indicates an elevated likelihood of a blood infection. Any value within this range may be considered as an appropriate threshold value. For example, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically two days after surgery and determining a level of at least one biomarker comprises determining the level of proADM or fragment(s) thereof and a level of proADM or fragment(s) thereof above a threshold value±20% or more selected from a range of values in between 1.2 nmol/L and 1.8 nmol/L indicates an elevated likelihood of a blood infection. Any value within this range may be considered as an appropriate threshold value. For example, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.2 nmol/L and 1.8 nmol/L indicates an elevated likelihood of a blood infection. Any value within this range may be considered as an appropriate threshold value. For example, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically and determining a level of at least one biomarker comprises determining the level of proADM or fragment(s) thereof and a level of proADM or fragment(s) thereof above a threshold value±20% or more selected from a range of values in between 1.1 nmol/L and 1.5 nmol/L indicates an elevated likelihood of a blood infection. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample is isolated postsurgically at one day, two days, three days or more after surgery and determining a level of at least one biomarker comprises determining the level of proADM or fragment(s) thereof and a level of proADM or fragment(s) thereof above a threshold value±20% or more selected from a range of values in between 1.3 nmol/L and 1.8 nmol/L indicates an elevated likelihood of a blood infection. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment a first sample is isolated from the patient postsurgically at a first point in time, preferably one day or more after surgery, and a second sample is isolated from the patient postsurgically at a subsequent second point in time, wherein preferably between the first and second point in time at least 12 hours, 24 hours, 36 hours, 48 hours or more have passed and determining a level of at least one biomarker comprises determining the level of proADM or fragment(s) thereof and an increase or a leveling in the level of proADM or fragment(s) thereof indicates an elevated likelihood of a blood infection.

In one embodiment a first sample is isolated from the patient postsurgically at a first point in time, preferably one day or more after surgery, and a second sample is isolated from the patient postsurgically at a subsequent second point in time, wherein preferably between the first and the second point in time at least 12 hours, 24 hours, 36 hours, 48 hours or more have passed and determining a level of at least one biomarker comprises determining the level of proADM or fragment(s) thereof and an increase of the level of proADM or fragment(s) thereof above a threshold value±20% or more selected from a range of values in between 0.01 nmol/L and 0.4 nmol/L indicates an elevated likelihood of a blood infection. Any value within this range may be considered as an appropriate threshold value for the increase of proADM or fragment(s) in between the first and second point in time. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4 nmol/L.

PCT/Any infection:

In one embodiment determining a level of at least one additional biomarker comprises determining the level of PCT or fragment(s) thereof and a level of PCT or fragment(s) thereof above a threshold value indicates an elevated likelihood of an infection.

In one embodiment the sample is isolated postsurgically within 12 hours, preferably within 6 hours after surgery and determining a level of at least one biomarker comprises determining the level of PCT or fragment(s) thereof and a level of PCT or fragment(s) thereof above a threshold value±20% or more selected from a range of values in between 0.2 μg/L and 0.3 μg/L indicates an elevated likelihood of an infection. Any value within this range may be considered as an appropriate threshold value. For example, 0.1, 0.2, 0.21, 0.22, 0.23, 0.24, 0.25, 0.26, 0.27, 0.28, 0.29, 0.3 μg/L.

In one embodiment the sample is isolated postsurgically one day after surgery and determining a level of at least one biomarker comprises determining the level of PCT or fragment(s) thereof and a level of PCT or fragment(s) thereof above a threshold value±20% or more selected from a range of values in between 0.6 μg/L and 0.9 μg/L indicates an elevated likelihood of an infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9 μg/L.

In one embodiment the sample is isolated postsurgically two days after surgery and determining a level of at least one biomarker comprises determining the level of PCT or fragment(s) thereof and a level of PCT or fragment(s) thereof above a threshold value±20% or more selected from a range of values in between 0.7 μg/L and 1.3 μg/L indicates an elevated likelihood of an infection. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, μg/L.

In one embodiment the sample is isolated postsurgically three days after surgery and determining a level of at least one biomarker comprises determining the level of PCT or fragment(s) thereof and a level of PCT or fragment(s) thereof above a threshold value±20% or more selected from a range of values in between 0.6 μg/L and 1.3 μg/L indicates an elevated likelihood of an infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 μg/L.

In one embodiment the sample is isolated postsurgically at one day or more after surgery and determining a level of at least one biomarker comprises determining the level of PCT or fragment(s) thereof and a level of PCT or fragment(s) thereof above a threshold value±20% or more selected from a range of values in between 0.6 μg/L and 1.1 μg/L indicates an elevated likelihood of an infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1 μg/L.

In one embodiment the sample is isolated postsurgically at two days, three days or more after surgery and determining a level of at least one biomarker comprises determining the level of PCT or fragment(s) thereof and a level of PCT or fragment(s) thereof above a threshold value±20% or more selected from a range of values in between 0.7 μg/L and 1.3 μg/L indicates an elevated likelihood of an infection. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 μg/L.

In one embodiment a first sample is isolated from the patient postsurgically at a first point in time, preferably one day or more after surgery, and a second sample is isolated from the patient postsurgically at a subsequent second point in time, wherein preferably between the first and second point in time at least 12 hours, 24 hours, 36 hours, 48 hours or more have passed and determining a level of at least one biomarker comprises determining the level of PCT or fragment(s) thereof and an increase in the level of PCT or fragment(s) thereof indicates an elevated likelihood of an infection.

In one embodiment a first sample is isolated from the patient postsurgically at a first point in time, preferably one day or more after surgery, and a second sample is isolated from the patient postsurgically at a subsequent second point in time, wherein preferably between the first and the second point in time at least 12 hours, 24 hours, 36 hours, 48 hours or more have passed and determining a level of at least one biomarker comprises determining the level of PCT or fragment(s) thereof and an increase of the level of PCT or fragment(s) thereof above a threshold value±20% or more selected from a range of values in between 0.1 μg/L and 0.6 μg/L indicates an elevated likelihood of an infection. Any value within this range may be considered as an appropriate threshold value for the increase of PCT or fragment(s) in between the first and second point in time. For example, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6 μg/L.

### PCT/Blood Infection:

In one embodiment determining a level of at least one additional biomarker comprises determining the level of PCT or fragment(s) thereof and a level of PCT or fragment(s) thereof above a threshold value indicates an elevated likelihood of a blood infection.

In one embodiment the sample is isolated postsurgically within 12 hours, preferably within 6 hours after surgery and determining a level of at least one biomarker comprises determining the level of PCT or fragment(s) thereof and a level of PCT or fragment(s) thereof above a threshold value±20% or more selected from a range of values in between 0.1 μg/L and 0.3 μg/L indicates an elevated likelihood of a blood infection. Any value within this range may be considered as an appropriate threshold value. For example, 0.1, 0.11, 0.12, 0.13, 0.14, 0.15, 0.16, 0.17, 0.18, 0.19, 0.2, 0.2, 0.21, 0.22, 0.23, 0.24, 0.25, 0.26, 0.27, 0.28, 0.29, 0.3 μg/L.

In one embodiment the sample is isolated postsurgically one day after surgery and determining a level of at least one biomarker comprises determining the level of PCT or fragment(s) thereof and a level of PCT or fragment(s) thereof above a threshold value±20% or more selected from a range of values in between 0.6 μg/L and 1.8 μg/L indicates an elevated likelihood of an blood infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 μg/L.

In one embodiment the sample is isolated postsurgically two days after surgery and determining a level of at least one biomarker comprises determining the level of PCT or fragment(s) thereof and a level of PCT or fragment(s) thereof above a threshold value±20% or more selected from a range of values in between 0.7 μg/L and 2.3 μg/L indicates an elevated likelihood of a blood infection. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 μg/L.

In one embodiment the sample is isolated postsurgically three days after surgery and determining a level of at least one biomarker comprises determining the level of PCT or fragment(s) thereof and a level of PCT or fragment(s) thereof above a threshold value±20% or more selected from a range of values in between 0.7 μg/L and 2.3 μg/L indicates an elevated likelihood of a blood infection. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 μg/L.

In one embodiment the sample is isolated postsurgically at one day or more after surgery and determining a level of at least one biomarker comprises determining the level of PCT or fragment(s) thereof and a level of PCT or fragment(s) thereof above a threshold value±20% or more selected from a range of values in between 0.7 μg/L and 1.8 μg/L indicates an elevated likelihood of an blood infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 μg/L.

In one embodiment the sample is isolated postsurgically at two days, three days or more after surgery and determining a level of at least one biomarker comprises determining the level of PCT or fragment(s) thereof and a level of PCT or fragment(s) thereof above a threshold value±20% or more selected from a range of values in between 1 μg/L and 2.3 μg/L indicates an elevated likelihood of a blood infection. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 μg/L.

In one embodiment a first sample is isolated from the patient postsurgically at a first point in time, preferably one day or more after surgery, and a second sample is isolated from the patient postsurgically at a subsequent second point in time, wherein preferably between the first and second point in time at least 12 hours, 24 hours, 36 hours, 48 hours or more have passed and determining a level of at least one biomarker comprises determining the level of PCT or fragment(s) thereof and an increase in the level of PCT or fragment(s) thereof indicates an elevated likelihood of a blood infection.

In one embodiment a first sample is isolated from the patient postsurgically at a first point in time, preferably one day or more after surgery, and a second sample is isolated from the patient postsurgically at a subsequent second point in time, wherein preferably between the first and the second point in time at least 12 hours, 24 hours, 36 hours, 48 hours or more have passed and determining a level of at least one biomarker comprises determining the level of PCT or fragment(s) thereof and an increase of the level of PCT or fragment(s) thereof above a threshold value±20% or more selected from a range of values in between 0.1 μg/L and 1 μg/L indicates an elevated likelihood of a blood infection. Any value within this range may be considered as an appropriate threshold value for the increase of PCT or fragment(s) in between the first and second point in time. For example, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1 μg/L.

proET-1/any Infection:

In one embodiment determining a level of at least one additional biomarker comprises determining the level of proET-1 or fragment(s) thereof and a level of proET-1 or fragment(s) thereof above a threshold value indicates an elevated likelihood of an infection.

In one embodiment the sample is isolated postsurgically two days after surgery and determining a level of at least one biomarker comprises determining the level of proET-1 or fragment(s) thereof and a level of proET1 or fragment(s) thereof above a threshold value±20% or more selected from a range of values in between 85 pmol/L and 95 pmol/L indicates an elevated likelihood of an infection. Any value within this range may be considered as an appropriate threshold value. For example, 85, 86, 87, 88, 89, 90, 91, 92, 93, 94, 95 pmol/L.

In one embodiment the sample is isolated postsurgically three days after surgery and determining a level of at least one biomarker comprises determining the level of proET-1 or fragment(s) thereof and a level of proET1 or fragment(s) thereof above a threshold value±20% or more selected from a range of values in between 80 pmol/L and 95 pmol/L indicates an elevated likelihood of an infection. Any value within this range may be considered as an appropriate threshold value. For example, 80, 81, 82, 83, 84, 85, 86, 87, 88, 89, 90, 91, 92, 93, 94, 95 pmol/L.

In one embodiment the sample is isolated postsurgically two days, three days or more after surgery and determining a level of at least one biomarker comprises determining the level of proET-1 or fragment(s) thereof and a level of proET-1 or fragment(s) thereof above a threshold value±20% or more selected from a range of values in between 80 pmol/L and 95 pmol/L indicates an elevated likelihood of an infection. Any value within this range may be considered as an appropriate threshold value. For example, 80, 81, 82, 83, 84, 85, 86, 87, 88, 89, 90, 91, 92, 93, 94, 95 pmol/L.

proET-1/Blood Infection:

In one embodiment determining a level of at least one additional biomarker comprises determining the level of proET-1 or fragment(s) thereof and a level of proET-1 or fragment(s) thereof above a threshold value indicates an elevated likelihood of a blood infection.

In one embodiment the sample is isolated postsurgically two days after surgery and determining a level of at least one biomarker comprises determining the level of proET-1 or fragment(s) thereof and a level of proET1 or fragment(s) thereof above a threshold value±20% or more selected from a range of values in between 85 pmol/L and 105 pmol/L indicates an elevated likelihood of a blood infection. Any value within this range may be considered as an appropriate threshold value. For example, 85, 86, 87, 88, 89, 90, 91, 92, 93, 94, 95, 96, 97, 98, 99, 100, 101, 102, 103, 104, 105 pmol/L.

In one embodiment the sample is isolated postsurgically three days after surgery and determining a level of at least one biomarker comprises determining the level of proET-1 or fragment(s) thereof and a level of proET1 or fragment(s) thereof above a threshold value±20% or more selected from a range of values in between 80 pmol/L and 110 pmol/L indicates an elevated likelihood of a blood infection. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 pmol/L.

In one embodiment the sample is isolated postsurgically two days, three days or more after surgery and determining a level of at least one biomarker comprises determining the level of proET-1 or fragment(s) thereof and a level of proET-1 or fragment(s) thereof above a threshold value±20% or more selected from a range of values in between 80 pmol/L and 110 pmol/L indicates an elevated likelihood of a blood infection. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 pmol/L.

### ADM/Infection and MACCE/MINS:

In one embodiment determining a level of at least one additional biomarker comprises determining the level of proADM or fragment(s) thereof and a level of proADM or fragment(s) thereof above a threshold value indicates an elevated likelihood of an infection, preferably a blood infection, and an adverse cardiovascular or cerebrovascular event, preferably a MACCE and/or a MINS.

In one embodiment the sample is isolated preoperatively and determining a level of at least one biomarker comprises determining the level of proADM or fragment(s) thereof and a level of proADM or fragment(s) thereof above a threshold value±20% or more selected from a range of values in between 0.7 nmol/L and 0.9 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.70, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9 nmol/L.

In one embodiment the sample is isolated postsurgically within 12 hours, preferably within 6 hours after surgery and determining a level of at least one biomarker comprises determining the level of proADM or fragment(s) thereof and a level of proADM or fragment(s) thereof above a threshold value±20% or more selected from a range of values in between 1.0 nmol/L and 1.6 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6 nmol/L.

In one embodiment the sample is isolated postsurgically one day after surgery and determining a level of at least one biomarker comprises determining the level of proADM or fragment(s) thereof and a level of proADM or fragment(s) thereof above a threshold value±20% or more selected from a range of values in between 1.3 nmol/L and 2.2 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2 nmol/L.

In one embodiment the sample is isolated postsurgically two days after surgery and determining a level of at least one biomarker comprises determining the level of proADM or fragment(s) thereof and a level of proADM or fragment(s) thereof above a threshold value±20% or more selected from a range of values in between 1.3 nmol/L and 2.3 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.3 nmol/L and 2.3 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 nmol/L.

In one embodiment the sample is isolated postsurgically and determining a level of at least one biomarker comprises determining the level of proADM or fragment(s) thereof and a level of proADM or fragment(s) thereof above a threshold value±20% or more selected from a range of values in between 1.3 nmol/L and 2 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.3 nmol/L and 2.3 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 nmol/L.

In one embodiment a first sample is isolated from the patient postsurgically at a first point in time, preferably one day or more after surgery, and a second sample is isolated from the patient postsurgically at a subsequent second point in time, wherein preferably between the first and second point in time at least 12 hours, 24 hours, 36 hours, 48 hours or more have passed and determining a level of at least one biomarker comprises determining the level of proADM or fragment(s) thereof and an increase in the level of proADM or fragment(s) thereof indicates an elevated likelihood an infection, preferably a blood infection, and an adverse cardiovascular or cerebrovascular event, preferably a MACCE and/or a MINS.

In one embodiment a first sample is isolated from the patient postsurgically at a first point in time, preferably one day or more after surgery, and a second sample is isolated from the patient postsurgically at a subsequent second point in time, wherein preferably between the first and the second point in time at least 12 hours, 24 hours, 36 hours, 48 hours or more have passed and determining a level of at least one biomarker comprises determining the level of proADM or fragment(s) thereof and an increase of the level of proADM or fragment(s) thereof above a threshold value±20% or more selected from a range of values in between 0.01 nmol/L and 1 nmol/L indicates an elevated likelihood an infection, preferably a blood infection, and an adverse cardiovascular or cerebrovascular event, preferably a MACCE and/or a MINS. Any value within this range may be considered as an appropriate threshold value for the increase of proADM or fragment(s) in between the first and second point in time. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1 nmol/L.

### PCT/Infection and MACCE/MINS:

In one embodiment determining a level of at least one additional biomarker comprises determining the level of PCT or fragment(s) thereof and a level of PCT or fragment(s) thereof above a threshold value indicates an elevated likelihood of an infection, preferably a blood infection, and an adverse cardiovascular or cerebrovascular event, preferably a MACCE and/or a MINS.

In one embodiment the sample is isolated postsurgically within 12 hours, preferably within 6 hours after surgery and determining a level of at least one biomarker comprises determining the level of PCT or fragment(s) thereof and a level of PCT or fragment(s) thereof above a threshold value±20% or more selected from a range of values in between 0.1 μg/L and 0.4 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4 μg/L.

In one embodiment the sample is isolated postsurgically one day after surgery and determining a level of at least one biomarker comprises determining the level of PCT or fragment(s) thereof and a level of PCT or fragment(s) thereof above a threshold value±20% or more selected from a range of values in between 0.6 μg/L and 1.8 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 μg/L.

In one embodiment the sample is isolated postsurgically two days after surgery and determining a level of at least one biomarker comprises determining the level of PCT or fragment(s) thereof and a level of PCT or fragment(s) thereof above a threshold value±20% or more selected from a range of values in between 0.6 μg/L and 3 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3 μg/L.

In one embodiment the sample is isolated postsurgically three days after surgery and determining a level of at least one biomarker comprises determining the level of PCT or fragment(s) thereof and a level of PCT or fragment(s) thereof above a threshold value±20% or more selected from a range of values in between 0.6 μg/L and 3 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3 μg/L.

In one embodiment the sample is isolated postsurgically and determining a level of at least one biomarker comprises determining the level of PCT or fragment(s) thereof and a level of PCT or fragment(s) thereof above a threshold value±20% or more selected from a range of values in between 0.7 μg/L and 3 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3 μg/L.

In one embodiment the sample is isolated postsurgically at one day, two days, three days or after surgery more and determining a level of at least one biomarker comprises determining the level of PCT or fragment(s) thereof and a level of PCT or fragment(s) thereof above a threshold value±20% or more selected from a range of values in between 1 μg/L and 3 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3 μg/L.

In one embodiment a first sample is isolated from the patient postsurgically at a first point in time, preferably one day or more after surgery, and a second sample is isolated from the patient postsurgically at a subsequent second point in time, wherein preferably between the first and second point in time at least 12 hours, 24 hours, 36 hours, 48 hours or more have passed and determining a level of at least one biomarker comprises determining the level of PCT or fragment(s) thereof and an increase or a leveling in the level of PCT or fragment(s) thereof indicates an elevated likelihood of an infection, preferably a blood infection, and an adverse cardiovascular or cerebrovascular event, preferably a MACCE and/or a MINS.

In one embodiment a first sample is isolated from the patient postsurgically at a first point in time, preferably one day or more after surgery, and a second sample is isolated from the patient postsurgically at a subsequent second point in time, wherein preferably between the first and the second point in time at least 12 hours, 24 hours, 36 hours, 48 hours or more have passed and determining a level of at least one biomarker comprises determining the level of PCT or fragment(s) thereof and an increase of the level of PCT or fragment(s) thereof above a threshold value±20% or more selected from a range of values in between 0.1 μg/L and 2 μg/L indicates an elevated likelihood of an infection, preferably a blood infection, and an adverse cardiovascular or cerebrovascular event, preferably a MACCE and/or a MINS. Any value within this range may be considered as an appropriate threshold value for the increase of PCT or fragment(s) in between the first and second point in time. For example, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2 μg/L.

proET-1/Infection and MACCE/MINS:

In one embodiment determining a level of at least one additional biomarker comprises determining the level of proET-1 or fragment(s) thereof and a level of proET-1 or fragment(s) thereof above a threshold value indicates an elevated likelihood of an infection, preferably a blood infection, and an adverse cardiovascular or cerebrovascular event, preferably a MACCE and/or a MINS.

In one embodiment the sample is isolated preoperatively and determining a level of at least one biomarker comprises determining the level of proET-1 or fragment(s) thereof and a level of proET-1 or fragment(s) thereof above a threshold value±20% or more selected from a range of values in between 60 pmol/L and 85 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 60, 61, 62, 63, 64, 65, 66, 67, 68, 69, 70, 71, 72, 73, 74, 75, 76, 77, 78, 79, 80, 81, 82, 83, 84, 85 pmol/L.

In one embodiment the sample is isolated postsurgically within 12 hours, preferably within 6 hours after surgery and determining a level of at least one biomarker comprises determining the level of proET-1 or fragment(s) thereof and a level of proET-1 or fragment(s) thereof above a threshold value±20% or more selected from a range of values in between 80 pmol/L and 110 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 pmol/L.

In one embodiment the sample is isolated postsurgically one day after surgery and determining a level of at least one biomarker comprises determining the level of proET-1 or fragment(s) thereof and a level of proET1 or fragment(s) thereof above a threshold value±20% or more selected from a range of values in between 80 pmol/L and 120 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 pmol/L.

In one embodiment the sample is isolated postsurgically two days after surgery and determining a level of at least one biomarker comprises determining the level of proET-1 or fragment(s) thereof and a level of proET1 or fragment(s) thereof above a threshold value±20% or more selected from a range of values in between 80 pmol/L and 120 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 pmol/L.

In one embodiment the sample is isolated postsurgically three days after surgery and determining a level of at least one biomarker comprises determining the level of proET-1 or fragment(s) thereof and a level of proET1 or fragment(s) thereof above a threshold value±20% or more selected from a range of values in between 80 pmol/L and 120 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 pmol/L.

In one embodiment the sample is isolated postsurgically and determining a level of at least one biomarker comprises determining the level of proET-1 or fragment(s) thereof and a level of proET-1 or fragment(s) thereof above a threshold value±20% or more selected from a range of values in between 80 pmol/L and 110 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 pmol/L.

In one embodiment the sample is isolated postsurgically at one day, two days, three days or more after surgery and determining a level of at least one biomarker comprises determining the level of proET-1 or fragment(s) thereof and a level of proET-1 or fragment(s) thereof above a threshold value±20% or more selected from a range of values in between 80 pmol/L and 120 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 pmol/L.

In one embodiment a first sample is isolated from the patient postsurgically at a first point in time, preferably one day or more after surgery, and a second sample is isolated from the patient postsurgically at a subsequent second point in time, wherein preferably between the first and the second point in time at least 12 hours, 24 hours, 36 hours, 48 hours or more have passed and determining a level of at least one biomarker comprises determining the level of proET-1 or fragment(s) thereof and an increase or a leveling in the level of proET-1 or fragment(s) thereof indicates an elevated likelihood of an infection, preferably a blood infection, and an adverse cardiovascular or cerebrovascular event, preferably a MACCE and/or a MINS.

In one embodiment a first sample is isolated from the patient postsurgically at a first point in time, preferably one day or more after surgery, and a second sample is isolated from the patient postsurgically at a subsequent second point in time, wherein preferably between the first and the second point in time at least 12 hours, 24 hours, 36 hours, 48 hours or more have passed and determining a level of at least one biomarker comprises determining the level of proET-1 or fragment(s) thereof and an increase of the level of proET-1 or fragment(s) thereof above a threshold value±20% or more selected from a range of values in between 1 pmol/L and 40 pmol/L indicates an elevated likelihood of an infection, preferably a blood infection, and an adverse cardiovascular or cerebrovascular event, preferably a MACCE and/or a MINS. Any value within this range may be considered as an appropriate threshold value for the increase of proET-1 or fragment(s) in between the first and second point in time. For example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 pmol/L.

There is also an embodiment relating to determining, as an additional biomarker, a level of proET-1, PCT or proADM or fragment(s) thereof for diagnosing or predicting a cardiovascular or cerebrovascular event, which is a perioperative myocardial injury (PMI), and an infection, such as a blood infection:

In one embodiment determining a level of at least one additional biomarker comprises determining the level of proET-1, PCT and/or proADM or fragment(s) thereof, and a level of proET-1, PCT and/or proADM or fragment(s) thereof above a threshold value indicates an elevated likelihood of an adverse cardiovascular or cerebrovascular event, preferably a perioperative myocardial injury (PMI), and the presence of an infection, preferably a blood infection. The sample may in some embodiments be obtained preoperatively or during surgery.

The invention also pertains to a kit for carrying out the method as explained herein. All of the features, effects and advantages of the inventive method also apply to the inventive kit and vice versa. To avoid repetitions, reference is therefore made to the previous explanations. Specifically, the inventive kit comprises at least one detection reagent for determining the level of at least one of the biomarkers copeptin, troponin and brain natriuretic peptide (BNP), in a sample of a bodily fluid from a patient, and reference data from patients who underwent gastrointestinal surgery, particularly reference levels, corresponding to at least one of copeptin, tro-ponin and BNP levels, wherein said reference data is preferably stored on a computer-readable medium and/or employed in the form of computer-executable code configured for comparing the determined level of at least one of copeptin, troponin and BNP to said reference data. Especially preferably, the detection reagents comprise reagents for determining the levels of the biomarkers copeptin, troponin and brain natriuretic peptide (BNP) in a sample of a bodily fluid from said patient, and wherein the reference data comprises data corresponding to said biomarkers in patients who underwent gastrointestinal surgery.

The detection reagents can for example comprise capture molecules. They can, for example, be designed for use in an assay system such as the Thermo Scientific B⋅R⋅A⋅H⋅M⋅S Kryptor Compact plus. In a preferred embodiment of the kit, the detection reagents comprise also reagents for determining the level of at least one of the biomarkers mid-regional proadrenomedullin (MR-proADM), C-terminal proendothelin-1 (CT-proET-1) and procalcitonin (PCT) in a sample of a bodily fluid from said patient, and wherein the reference data comprise data corresponding to said at least one biomarker in patients who underwent gastrointestinal surgery.

Herein, the term “capture molecules” comprises molecules which may be used to bind target molecules or molecules of interest, i.e. analytes (e.g. the biomarkers), from a sample. Capture molecules must thus be shaped adequately, both spatially and in terms of surface features, such as surface charge, hydrophobicity, hydrophilicity, presence or absence of Lewis donors and/or acceptors, to specifically bind the target molecules or molecules of interest. Hereby, the binding may, for instance, be mediated by ionic, van-der-Waals, pi-pi, sigma-pi, hydrophobic or hydrogen bond interactions or a combination of two or more of the aforementioned interactions or covalent interactions between the capture molecules and the target molecules or molecules of interest. In the context of the present invention, capture molecules may for instance be selected from the group consisting of a nucleic acid molecule, a carbohydrate molecule, a PNA molecule, a protein, a peptide and a glycoprotein. Capture molecules include, for example, antibodies, aptamers, DARpins (Designed Ankyrin Repeat Proteins). Affimers and the like are included.

The term “antibody” as used herein, unless indicated otherwise, is used broadly to refer to both, antibody molecules such as monoclonal antibodies, polyclonal antiserum, enriched or purified polyclonal antibodies, recombinant antibodies and a variety of antibody-derived molecules, specifically functional derivatives. Such antibody-derived molecules comprise at least one variable region (either a heavy chain or a light chain variable region), as well as individual antibody light chains, individual antibody heavy chains, chimeric fusions between antibody chains and other molecules, and the like. Functional immunoglobulin fragments according to the present invention may be Fv, scFv, disulfide-linked Fv, Fab, and F(ab′)2. Also encompassed by the term “antibody” are polyclonal antibodies, monoclonal antibodies, preferably IgG1 antibodies; chimeric monoclonal antibodies; humanized antibodies, genetically engineered monoclonal antibodies. Functional derivatives are chemically and/or biochemically modified variants of the antibodies/antisera having an analogous functionality/binding capacity.

The reference data as provided in the inventive kit is preferably designed to differentiate a patient between at least one of the pairs of having/not having and developing/not developing a MACCE or MINS after gastrointestinal surgery. The reference data is therefore preferably designed to be compared to the biomarker levels determined in samples taken from the patient at specific points in time as described above. Through the use of the inventive kit, a clinician may reach a quick and easy diagnosis and/or prognosis of a patient undergoing gastrointestinal surgery in view of the risk of having and/or developing a MACCE or MINS.

The invention also encompasses a computer running computer-executable code, with the computer-executable code being configured to carry out steps iv) and/or v) of the inventive method. For example, the computer-executable code is designed to create the combined assessment from input data, for example, the determined biomarker levels. In a preferred embodiment, the computer running the code is also connected to an assay system, the assay system being configured to carry out step ii) and, optionally, step iii) of the inventive method. The assay system can be, for example, a Thermo Scientific B⋅R⋅A⋅H⋅M⋅S Kryptor Compact plus. In this way, the assay system can provide the computer with the input data for carrying out steps iv) and/or v). The computer can also be provided with the aforementioned reference data. Optimally, a user merely has to carry out step i), while the computer with the assay system carry out the rest of the method and present the user with the resulting diagnosis and/or prognosis, or at least the score(s) on the basis of which the diagnosis and/or prognosis can be made.

Finally, the invention also relates to a computer program product comprising a set of computer instructions stored on at least one computer-readable medium, wherein said set of computer instructions further comprises instructions executable by one or more processors to carry out steps iv) and/or v) of the method of the invention. The computer-readable medium may be any kind of computer storage means, such as a USB stick, an external or internal hard disk, a diskette, a storage tape, an external handheld device, such as a mobile phone, tablet, or the like.

### BRIEF DESCRIPTION OF THE DRAWINGS

In the drawings, FIGS. 1-6 show the determined levels of the biomarkers copeptin (marked COP or COPAVP), cTnT (marked hsTnT for a high-sensitivity assay used, see below), NT-proBNP, MR-proADM, CTproET-1 and PCT in a patient population at different times of sampling and divided in patients who did and did not develop a MACCE.

FIGS. 7-12 show the determined levels of the biomarkers in analogy to FIGS. 1-6, wherein the patient population is divided in patients who did and did not develop a MINS.

FIGS. 13-18 show the determined levels of the biomarkers in analogy to FIGS. 1 to 6, wherein the patient population is divided in patients who did and did not develop an infection in addition to MACCE.

FIGS. 19-21 show the determined levels of MR-proADM, PCT and CT-proET-1, wherein the patient population is divided in patients who did and did not develop an infection.

FIGS. 22-24 show the determined levels of MR-proADM, PCT and CT-proET-1, wherein the patient population is divided in patients who did and did not develop a blood infection.

FIGS. 22-24 show the determined levels of MR-proADM, PCT and CT-proET-1, at different points in time, differentiated between different combinations of MACCE and infection.

### EXAMPLE

A multicenter, prospective cohort study in several Swedish hospitals was conducted. Subjects were recruited consecutively between April 2017 and October 2018. Patients aged 50 or older, undergoing elective abdominal surgery rated as major or major/complex by SORT, Surgical Outcome Risk Tool [Protopapa et al., British Journal of Surgery, 101(13), pp. 1774-1783. doi: 10.1002/bjs.9638, 2014], were eligible. The procedure also had to be performed under general anesthesia and require at least one overnight stay. Eligible patients were identified by screening daily patient lists in preoperative assessment clinics. Patients were informed of the study objectives, methods, expected benefits, potential dangers and the possibility of withdrawal at any time before consenting to participate. Informed written consent was obtained from all patients before being included. Participants could be included only once and patients were excluded if they were not able to give an informed consent or underwent one of the following types of abdominal surgery: transplantation, trauma, endocrine, vascular or endovascular. Included patients were excluded if the elective surgery was cancelled because the patient died, or if another non-elective surgery was performed prior to the elective one. The predefined study endpoint was MACCE (as defined above) within 30 days. Overall patient characteristics are given in Table 1.

TABLE 1
Overall patient characteristics

| | | No | | No | | No | Any | No Blood | Blood |
|---|---|---|---|---|---|---|---|---|---|
| | All | MACCE | MACCE | MINS | MINS | Infection | Infection | Infection | Infection |
| No. of | 387 | 350 | 37 | 293 | 94 | 282 | 105 | 376 | 11 |
| Patients | | | | | | | | | |
| Avg Age | 70 | 70 | 72 | | | 70 | 69 | 70 | 72 |
| Women | 44% | 46% | 30% | | | 44% | 45% | 44% | 45% |
| Blood | 11 | 6 | 5 | 10 | 1 | 0 | 11 | 0 | 11 |
| infection | (2.8%) | | | | | | | | |
| Any | 105 | 88 | 17 | 73 | 32 | 0 | 105 | 94 | 11 |
| infection | (27%) | | | | | | | | |
| ASA I-II | 265 | 243 | 21 | | | 199 | 66 | 260 | 5 |
| | (68%) | | | | | | | | |
| ASA | 116 | 100 | 16 | | | 79 | 37 | 110 | 6 |
| III-IV | (30%) | | | | | | | | |
| MINS | 94 | 80 | 14 | 0 | 94 | 62 | 32 | 93 | 1 |
| | (24%) | | | | | | | | |
| MACCE | 37 | 0 | 37 | 23 | 14 | 20 | 17 | 32 | 5 |
| | (9.6%) | | | | | | | | |
| Deceased | 5 | 0 | 5 | | | 0 | 5 | 3 | 2 |
| | (1.3%) | | | | | | | | |

Five sample points were defined: preoperatively (<24 h prior to anesthesia), postoperatively (within 24 hours of surgery) and on days 1, 2 and 3 after surgery. At these times, an arterial or venous blood sample was obtained using an 8.5 ml EDTA Vacutainer®. Blood samples were sent to the local clinical chemistry laboratory where they were centrifuged, plasma was frozen in aliquots at −80° C. and stored until batch-analyses of plasma concentration of the biomarkers were performed. Simultaneously, an ECG was taken from each patient at each sampling point.

The occurrence of MACCE was reviewed on day 1, 2 and 3 through medical charts and at 30 days after surgery via a telephone interview and/or by consulting the patient's medical records. The incidence of MINS was calculated from analyzed cTnT plasma levels [according to Sessler and Khanna; Intensive Care Med (2018) 44:811-822, https://doi.org/10.1007/s00134-018-5224-7]. The mortality rate was assessed using the Swedish population register. If a patient was discharged, dropped out or died before all samples had been obtained, the patient was not excluded and the collected samples were analyzed unless the patient asked not to be included in the data analyses.

Copeptin, MR-proADM, CT-proET-1 and PCT were measured using a Thermo Scientific B⋅R⋅A⋅H⋅M⋅S Kryptor Compact plus according to the instructions of the manufacturer (B⋅R⋅A⋅H⋅M⋅S GmbH, Hennigsdorf, Germany). Cardiac troponin T (cTnT) and NT-proBNP were measured using a Cobas e 602/Cobas e 601/Cobas e 411 assay according to the instructions of the manufacturer (Roche Diagnostics, Mannheim, Germany).

Data analyses were performed using Graphpad PRISM 8. Data distribution was reviewed using D'Agostino & Pearson test. Comparisons between different groups were performed using Fisher's exact test and Kruskal Wallis test. P-values<0.05 were considered significant.

Patient- and clinical characteristics are presented in Table 2, wherein ASA stands for the physical status classification system according to the American Society of Anesthesiologists (ASA). 387 surgical patients were recruited and included in the analyses. It is noteworthy that only 43% of patients who suffered from MACCE were classified as high-risk patients (ASA III-IV), and only 38% were classified as having MINS.

TABLE 2
Patient and clinical characteristics for patients with and without MACCE.

| | All | No MACCE | MACCE |
|---|---|---|---|
| No. of patients | 387 | 350 | 37 (9.6%) |
| Age median | 70 | 70 | 72 |
| Women | 44% | 46% | 30% |
| ASA I-II | 265 | 243 | 21 |
| ASA III-IV | 116 (30%) | 100 (29%) | 16 (43%) |
| MINS | 94 (24%) | 80 (23%) | 14 (38%) |
| Deceased | 5 (1.3%) | 0 | 5 (13.5%) |

The collected data did not pass the D'Agostino-Pearson normality test, therefore non-parametric tests were used in the analyses. Comparison of how plasma levels of the measured biomarkers varied between patients affected, and not affected, by MACCE was performed using a Kruskal Wallis test. FIGS. 1-6 show plasma levels of the measured biomarkers copeptin, cTnT, NT-proBNP, MR-proADM, CT-proET-1 and PCT in patients with and without MACCE at all sampling points/times. In the figures, PreOp designates the preoperative sampling point, PACU (post anesthesia care unit) designates the postoperative sampling point, POD1 designates the sampling point one day after surgery, POD2 designates the sampling point two days after surgery and POD3 designates the sampling point three days after surgery.

As can be seen from FIGS. 1-6, the biomarker levels of all measured biomarkers are increased in the group of patients who suffered a MACCE within 30 days from gastrointestinal surgery in comparison with the group of patients not suffering a MACCE.

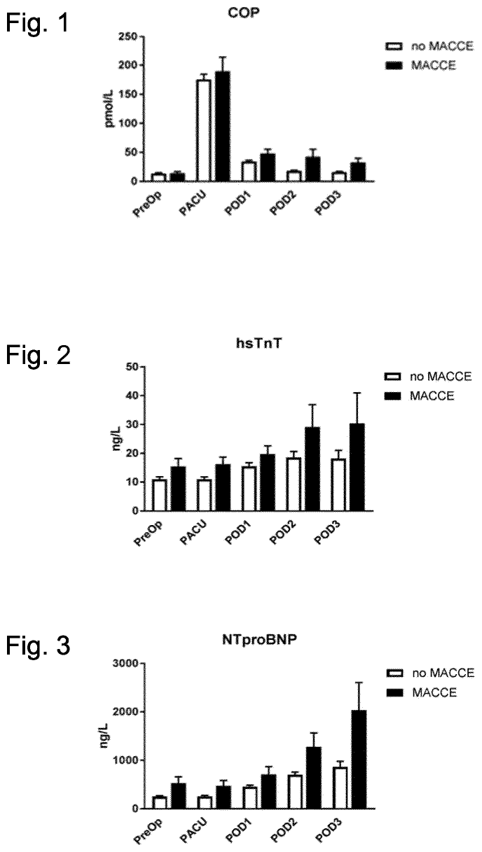

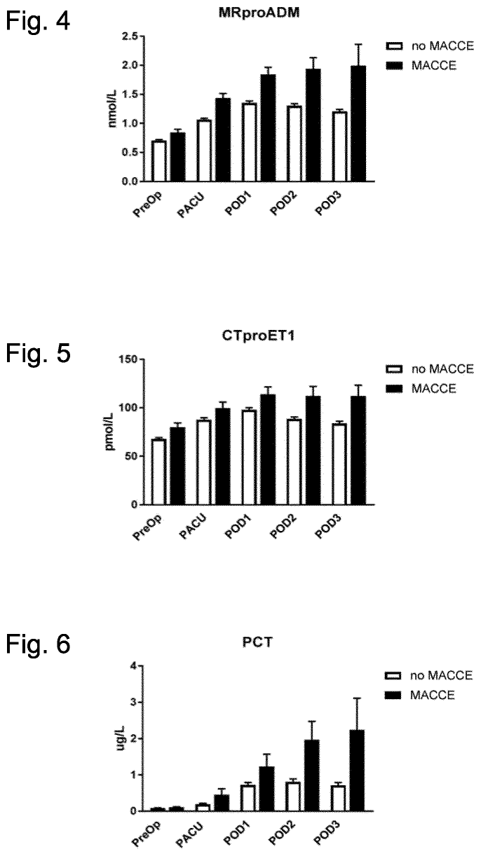

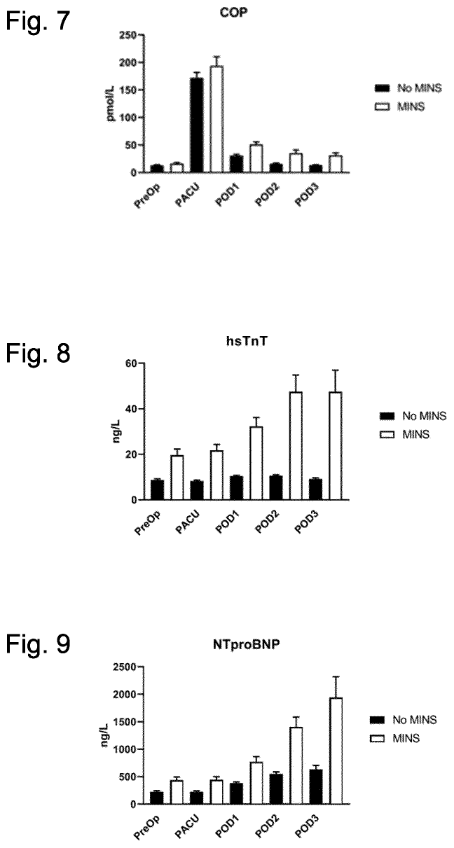

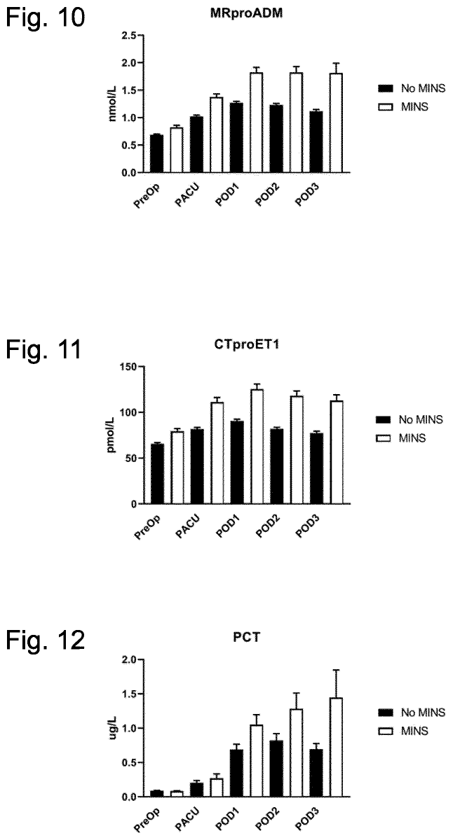

The variation of the biomarker levels was also compared in patients with and without MINS using a Kruskal Wallis test. In analogy to FIGS. 1-6, FIGS. 7-12 show plasma levels of the measured biomarkers copeptin, cTnT, NT-proBNP, MR-proADM, CT-proET-1 and PCT in patients with and without MINS at all sampling points/times. Again, as can be seen from FIGS. 7-12, the biomarker levels of all measured biomarkers are increased in the group of patients who suffered a MINS within 30 days from gastrointestinal surgery in comparison with the group of patients not suffering a MINS.

With regard to an infection as a postsurgical adverse event 105 (27%) patients developed an infection of which 11 suffered of a blood infection. The overall mortality rate was 1.3% (5 deceased). Table 2 summarizes the patient characteristics in regard to the occurrence of an infection. Biomarkers levels were determined for all patients preoperatively (preOP), on the post-anesthesia care unit (PACU) as well as one day (POD1), two days (POD2) and three days (POD3) after surgery.

TABLE 3
Patient characteristics in regards to the incidence of any infection

| | All | No Infection | Any Infection |
|---|---|---|---|
| No. of Patients | 387 | 282 | 105 (27%) |
| Avg Age | 70 | 70 | 69 |
| Women | 44% | 44% | 45% |
| ASA I-II | 265 | 199 | 66 |
| ASA III-IV | 116 (30%) | 79 (28%) | 37 (35%) |
| MINS | 94 (24%) | 62 (22%) | 32 (30.5%) |
| MACCE | 37 (9.6%) | 20 (7.1%) | 17 (16.2%) |
| Deceased | 5 (1.3%) | 0 (0%) | 5 (4.8%) |

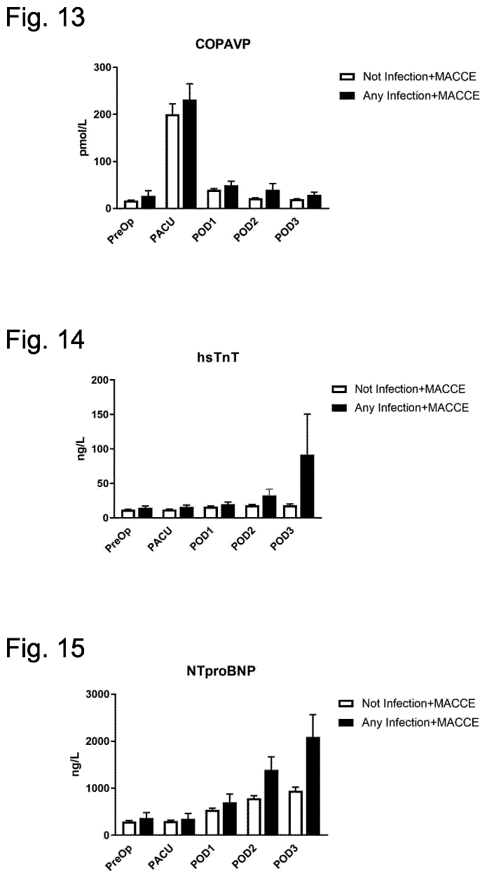

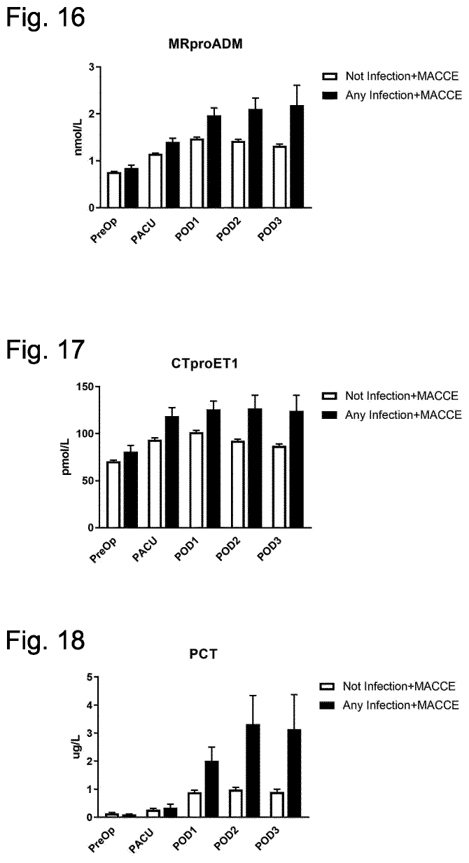

FIG. 13-18 show biomarker levels of patients with MACCE with or without an additional infection. All of the biomarkers show significantly higher levels in patients with a combination of MACCE and an infection, with the exception of PCT before the surgery. With the biomarkers troponin (FIG. 14), BNP (FIG. 15), proADM (FIG. 16), proET-1 (FIG. 17) and PCT (FIG. 18), the increase in biomarker level becomes even more pronounced at later points in time (i.e. POD2 and POD3), while copeptin (FIG. 13) shows a more constant increase compared with patients without infections over all points in time analysed. All of the biomarkers are therefore suitable for the differentiation between patients with MACCE alone and patients with both MACCE and an infection. The underlying data is presented in Table 4.

TABLE 4
Biomarker values for patients developing MACCE with or without infection

| | | PreOP | PACU | POD1 | POD2 | POD3 |
|---|---|---|---|---|---|---|
| Copeptin [pmol/L] | No inf + MACCE | 15 | 195 | 33 | 20 | 18 |
| | Inf + MACCE | 25 | 230 | 47 | 37 | 27 |
| hsTnT [ng/L] | No inf + MACCE | 9 | 8 | 14 | 15 | 15 |
| | Inf + MACCE | 12 | 14 | 23 | 30 | 90 |
| NT-proBNP [ng/L] | No inf + MACCE | 260 | 270 | 550 | 760 | 950 |
| | Inf + MACCE | 330 | 300 | 700 | 1330 | 2050 |
| MR-proADM [nmol/L] | No inf + MACCE | 0.74 | 1.12 | 1.48 | 1.42 | 1.28 |
| | Inf + MACCE | 0.83 | 1.40 | 1.93 | 2.09 | 2.20 |
| CT-proET-1 [pmol/L] | No inf + MACCE | 70 | 92 | 100 | 90 | 88 |
| | Inf + MACCE | 78 | 118 | 125 | 127 | 123 |
| PCT [μg/L] | No inf + MACCE | 0.06 | 0.10 | 0.83 | 0.98 | 0.85 |
| | Inf + MACCE | 0.04 | 0.14 | 1.98 | 3.29 | 3.09 |

From the values given in Table 4, the skilled person can easily infer which test parameters, for example, cut-off values, to use in the inventive method. These can then be chosen with regard to the actual application at hand. It also becomes clear that the measured biomarker levels should expediently be evaluated with respect to the point in time the samples were taken. For example, a copeptin level of above 15 pmol/L, preferably above 20 pmol/L or above 25 pmol/L, in a sample taken preoperatively (PreOP) is indicative of an increased risk of the patient developing both MACCE and an infection after surgery. At other points in time of sampling, these values may vary, and for example be above 195 pmol/L or above 200 pmol/L or above 205 pmol/L or above 210 pmol/L or above 215 pmol/L or above 220 pmol/L or above 225 pmol/L or above 230 pmol/L at PACU, above 30 pmol/L or above 35 pmol/L or above 40 pmol/L or above 45 pmol/L at POD1, above 20 pmol/L or above 25 pmol/L or above 30 pmol/L or above 35 pmol/L at POD2, above 15 pmol/L or above 20 pmol/L or above 25 pmol/L at POD3. Of course, for any biomarker evaluative values other than cut-off values may be calculated which relate the measured biomarker level to the biomarker level established for patients having MACCE without an infection and/or having no symptoms at all. For example, a factor may be established by which the measured biomarker level differs from the level of patients with MACCE but without an infection.

For hsTnT, a level of above 8 ng/L or above 9 ng/L or above 10 ng/L or above 11 ng/L or above 12 ng/L in a sample taken at PreOP and/or at PACU is indicative of an increased risk of the patient developing both MACCE and an infection after surgery. At other points in time of sampling, these values may vary, and, for example, be above 10 ng/L or above 15 ng/L or above 20 ng/L or above 25 ng/L or above 30 ng/L at POD1 and/or POD2 and/or POD3. A global cut-off value for all points in time could for example be chosen at above 10 ng/L or above 15 ng/L, depending on the desired sensitivity and specificity as well as other factors of the application at hand.

For NT-proBNP, a level of above 260 ng/L or above 280 ng/L or above 300 ng/L or above 320 ng/L in a sample taken at PreOP and/or at PACU is indicative of an increased risk of the patient developing both MACCE and an infection after surgery. At other points in time of sampling, these values may vary, and, for example, be above 500 ng/L or above 550 ng/L or above 600 ng/L or above 650 ng/L or above 700 ng/L or above 750 ng/L or above 800 ng/L or above 850 ng/L at POD1 and/or POD2 and/or POD3. A global cut-off value for all points in time could for example be chosen at above 300 ng/L or above 500 ng/L, depending on the desired sensitivity and specificity as well as other factors of the application at hand.

For MR-proADM, a level of above 0.70 nmol/L or above 0.75 nmol/L above 0.80 nmol/L in a sample taken at PreOP is indicative of an increased risk of the patient developing both MACCE and an infection after surgery. At other points in time of sampling, these values may vary, and, for example, be above 1.00 nmol/L or above 1.10 nmol/L or above 1.20 nmol/L or above 1.30 nmol/L or above 1.40 nmol/L or above 1.50 nmol/L or above 1.60 nmol/L at PACU and/or POD1 and/or POD2 and/or POD3. A global cut-off value for all points in time could, for example, be chosen at above 0.8 nmol/L or above 1.5 nmol/L, depending on the desired sensitivity and specificity as well as other factors of the application at hand.

For CT-proET-1, a level of above 65 pmol/L or above 70 pmol/L or above 75 pmol/L above 80 pmol/L in a sample taken at PreOP is indicative of an increased risk of the patient developing both MACCE and an infection after surgery. At other points in time of sampling, these values may vary, and, for example, be above 85 pmol/L or above 90 pmol/L or above 95 pmol/L or above 100 pmol/L or above 105 pmol/L or above 110 pmol/L or above 115 pmol/L or above 120 pmol/L or above 125 pmol/L at PACU and/or POD1 and/or POD2 and/or POD3. A global cut-off value for all points in time could, for example, be chosen at above 80 pmol/L or above 100 pmol/L, depending on the desired sensitivity and specificity as well as other factors of the application at hand.

For PCT, a level of above 0.06 μg/L or above 0.08 μg/L or above 0.10 μg/L or above 0.12 μg/L in a sample taken at PreOP and/or at PACU is indicative of an increased risk of the patient developing both MACCE and an infection after surgery. At other points in time of sampling, these values may vary, and, for example, be above 0.8 μg/L or above 0.9 μg/L or above 1.0 μg/L or above 1.1 μg/L or above 1.2 μg/L or above 1.3 μg/L or above 1.4 μg/L or above 1.5 μg/L at POD1 and/or POD2 and/or POD3. A global cut-off value for all points in time could, for example, be chosen at above 0.1 μg/L or above 1.0 μg/L or above 1.5 μg/L, depending on the desired sensitivity and specificity as well as other factors of the application at hand.

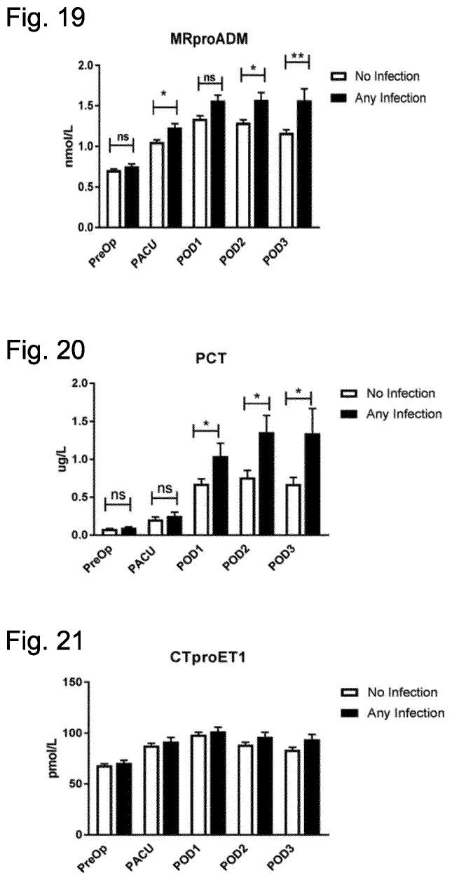

FIG. 19-21 summarize the results concerning infections for MR-proADM, PCT and CT-proET-1. The levels of all biomarkers, but in particular MR-proADM and PCT, were elevated in the group of patients that suffered an infection after surgery. This trend is visible for all sample points preOP, PACU, as well as PODs 1-3.

The level of MR-proADM for patients postsurgically developing an infection is elevated compared to the level in patients with no infection for sample points early on at PACU and even mildly preoperatively. The difference becomes more pronounced postsurgically from POD1 and in particular POD2 and POD3 (FIG. 19). The level of MR-proADM in patients without any infection falls after POD1 from approximately 1.25 nmol/L to below 1.2 nmol/L at POD3. In patients having an infection the level is steadily maintained above 1.5 nmol/L for all PODs 1-3.

For PCT as a marker for an occurrence of an infection a particular pronounced difference can be seen postsurgically from POD1 through POD3 (FIG. 20). While the level of PCT in postsurgical patients with no infection remains around or below 0.75 μg/L, the level of PCT in patients having an infection continuously rises from POD 1 (approximately 1.0 μg/L) to more than approximately 1.25 μg/L for PODs 2 and 3. Notably, also preoperatively and early on at PACU a mild difference is visible.

For CT-proET-1 the difference between patients with and without infections are less pronounced than for the markers PCT and MR-proADM. However, in particular at later sample points two or three days after surgery, a visible difference allows for a reliable distinction (FIG. 21).

The results demonstrate that in particular MR-proADM and PCT, but, to a somewhat lesser extent, also CT-proET-1 can be used for providing a reliable prediction or diagnosis for the incidence of infection in postsurgical patients and therefore can advantageously be combined with measurements of copeptin, troponin and BNP. The prediction or diagnosis can be established with a single threshold value for all sample points or at individual sample points (e.g. preOP, PACU, POD1-3) by comparison to sample point specific values.

In addition, the different temporal development of the levels of the biomarkers in patients with and without any infection allow for a further reliable prognosis or diagnosis, when taking samples at at least two points in time and analyzing the ratio or absolute difference for the different sample points.

Table 5 summarizes the patient characteristics in regard to the occurrence of a blood infection. Biomarkers levels were determined for all patients preoperatively (preOP), on the post-anesthesia care unit (PACU) as well as one day (POD1), two days (POD2) and three days (POD3) after surgery.

TABLE 5
Patient characteristics in regards to the incidence of a blood infection

| | All | No Blood Id | Blood Id |
|---|---|---|---|
| No. of Patients | 387 | 376 | 11 (2.8%) |
| Avg Age | 70 | 70 | 72 |
| Women | 44% | 44% | 45% |
| ASA I-II | 265 | 260 | 5 |
| ASA III-IV | 116 (30%) | 110 (29%) | 6 (55%) |
| MINS | 94 (24%) | 93 (25%) | 1 (9.1%) |
| MACCE | 37 (9.6%) | 32 (8.5%) | 5 (45.5%) |
| Deceased | 5 (1.3%) | 3 (0.8%) | 2 (18.2%) |

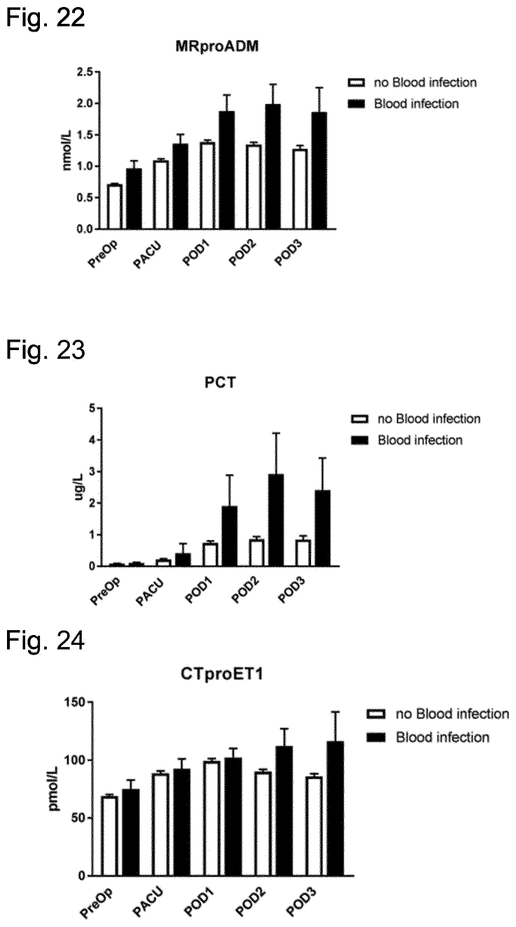

FIGS. 22-24 summarize the results for MR-proADM, PCT and CT-proET-1. The levels of all biomarkers, but in particular MR-proADM and PCT, were elevated in the group of patients that suffered a blood infection after surgery. This trend is visible for all sample points preOP, PACU, as well as PODs 1-3.

The level of MR-proADM for patients postsurgically developing a blood infection is elevated compared to the level in patients with no infection for sample points early on at PACU and notably even preoperatively. The difference becomes more pronounced postsurgically from POD1 to POD3 (FIG. 22). The level of MR-proADM in patients without any infection falls after POD1 from approximately 1.3 nmol/L to below 1.2 nmol/L at POD3. For patients having a blood infection the level is steadily maintained above approximately 1.75 nmol/L for all PODs 1-3.

Similar to the incident of an infection for the occurrence of a blood infection, a particularly pronounced difference in the level of PCT can be seen postsurgically from POD1 through POD3 (FIG. 23). While the level of PCT in postsurgical patients with no blood infection remains around or below 0.75 μg/L, the level of PCT in patients having a blood infection continuously rises from POD 1 (approximately 2.0 μg/L) to more than approximately 2.5 μg/L for PODs 2 and 3. Notably, also preoperatively and early on at PACU a mild difference is visible.

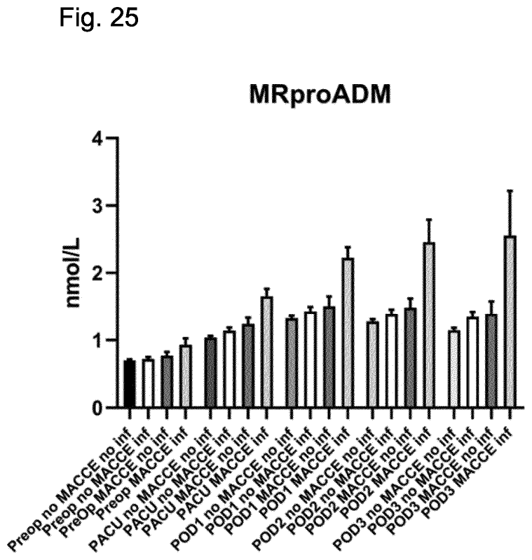

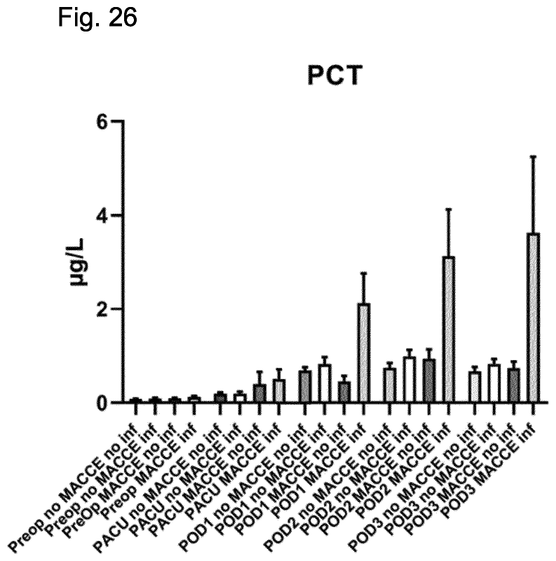

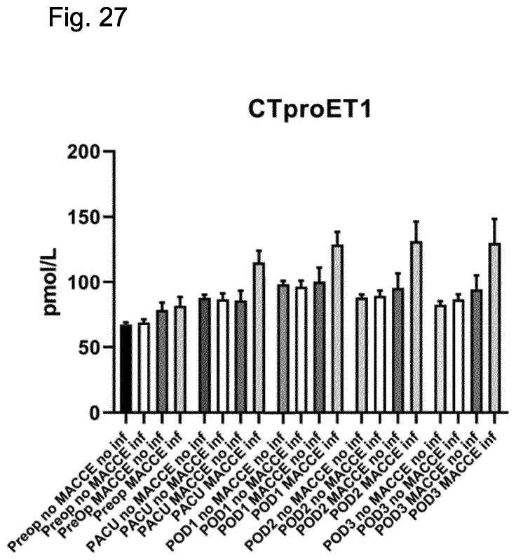

For CT-proET-1 the difference between patients with and without a blood infection are less pronounced than for the markers PCT and MR-proADM. However, in particular at later sample points two or three days after surgery, a visible difference allows for a distinction. While patients with a blood infection at POD2 and 3 had a level of CT-proET-1 of more than 100 pmol/L, the level of patients with no blood infection at POD2 and POD3 was around or below approximately 0.75 pmol/L (FIG. 24).

The results demonstrate that in particular MR-proADM and PCT, but, to a lesser extent, also CT-proET1 may serve for a reliable prediction or diagnosis for the incidence of a blood infection in postsurgical patients and therefore may advantageously be combined with measurements of copeptin, troponin and BNP. The prediction or diagnosis can be established with a single threshold value for all sample points or at individual sample points (e.g. preOP, PACU, POD1-3) by comparison to sample point specific values.

In addition, the different temporal developments of the levels of the biomarkers in patients with and without a blood infection allow for a further reliable prognosis or diagnosis when taking samples at two or more points of time and analyzing the ratio or absolute difference for the different sample points.

Table 6 summarizes the patient characteristics in regard to the postsurgical occurrence of an infection and MACCE. Biomarkers levels were determined for all patients preoperatively (preOP), on the post-anesthesia care unit (PACU) as well as one day (POD1), two days (POD2) and three days (POD3) after surgery.

TABLE 6
Patient characteristics in regards to the
incidence of an infection and MACCE

| No. of Patients | No infection | infection |
|---|---|---|
| No MACCE | 262 | 88 |
| MACCE | 20 | 17 |

FIGS. 25-27 summarize the results for MR-proADM, PCT and CT-proET-1.

Strikingly, the levels of all biomarkers were particularly elevated in the group of patients that suffered both an infection as well as a MACCE. This trend was clearly visible for all sample points preOP, PACU, as well as PODs 1-3.

For MR-proADM the level of patients that suffered of an infection and a MACCE exhibit clear differences compared to patients that did not suffer any of these events or that suffered of only one of the adverse events across all sample points (FIG. 25). The difference is particularly pronounced postsurgically. While patients that neither developed an infection nor experienced MACCE postsurgically showed a level of MR-proADM of 1 nmol/L or below at PACU or POD 1-3, patients that suffered both adverse events showed a steady increase in the level of MR-proADM from approximately 1.5 nmol/L at PACU to approximately 2.3 nmol/L at POD3.

PCT appears to be a particularly valuable marker for the combined prognosis or diagnosis of infections and MACCE. This is particularly the case for sample points postsurgically from POD1 to POD3 (FIG. 26). The PCT levels of patients that developed an infection and suffered a MACCE events at POD1 through POD3 were consistently above 2 μg/L, averaging on POD3 even at approximately 3.5 μg/L. On the contrary, for patients who postsurgically neither experienced a MACCE nor an infection, PCT levels of around or below 0.5 μg/L were determined. The more than 4-fold difference on POD1 and more than 7-fold difference on POD3 indicate a strong synergistic effect for the combined diagnosis or prediction of an infection plus MACCE using PCT as a marker.

Likewise, for CT-proET-1 across all sample points considerably higher levels were determined in patients suffering postsurgically both from an infection as well as a MACCE than in patients that experienced neither one of these adverse events (FIG. 27). While for the former group CT-proET-1 levels remained at or below approximately 80 pmol/L, in the latter group the postsurgical level of CT-proET-1 from PACU to POD3 was at or above approximately 125 pmol/L.

The data demonstrate that the prognosis and/or diagnosis of an infection and a MACCE using one of the described biomarkers can be achieved with a particularly high accuracy and reliability. These biomarkers may therefore advantageously be combined with measurements of copeptin, troponin and BNP. From the predictive potential of the markers for one of the adverse events such a strong predictive power could not have been expected, pointing towards a functional synergy of these biomarkers in regard to the diagnosis or prediction of these distinct adverse events.

The prediction or diagnosis can be established with a single threshold value for all sample points or at individual sample points (e.g. preOP, PACU, POD1-3) by comparison to sample point specific values.

In addition, the different temporal development of the level of the biomarkers in patients with and without an infection and a MACCE allow for a further reliable prognosis or diagnosis, when taking samples at two points in time or more and analyzing the ratio or absolute difference for the different sample points.

SEQUENCES
(amino acid sequence of pre-pro-AVP):
SEQ ID NO: 1

| | |
|---|---|
| 1 | MPDTMLPACF LGLLAFSSAC YFQNCPRGGK RAMSDLELRQ |
| | CLPCGPGGKG |
| 51 | RCFGPSICCA DELGCFVGTA EALRCQEENY LPSPCQSGQK |
| | ACGSGGRCAA |
| 101 | FGVCCNDESC VTEPECREGF HRRARASDRS NATQLDGPAG |
| | ALLLRLVQLA |
| 151 | GAPEPFEPAQ PDAY |

(amino acid sequence of pro-AVP):
SEQ ID NO: 2

| | |
|---|---|
| 1 | CYFQNCPRGG KRAMSDLELR QCLPCGPGGK GRCFGPSICC |
| | ADELGCFVGT |
| 51 | AEALRCQEEN YLPSPCQSGQ KACGSGGRCA AFGVCCNDES |
| | CVTEPECREG |
| 101 | FHRRARASDR SNATQLDGPA GALLLRLVQL AGAPEPFEPA |
| | QPDAY |

(amino acid sequence of CT-pre-proAVP
or copeptin):
SEQ ID NO: 3

| | |
|---|---|
| 1 | ASDRSNATQL DGPAGALLLR LVQLAGAPEP FEPAQPDAY |

(amino acid sequence of Neurophysin II):
SEQ ID NO: 4

| | |
|---|---|
| 1 | AMSDLELRQC LPCGPGGKGR CFGPSICCAD ELGCFVGTAE |
| | ALRCQEENYL |
| 51 | PSPCQSGQKA CGSGGRCAAF GVCCNDESCV TEPECREGFH |
| | RRA |

(amino acid sequence of cTnT (Isoform-6)):
SEQ ID NO: 5

| | |
|---|---|
| 1 | MSDIEEVVEE YEEEEQEEAA VEEQEEAAEE DAEAEAETEE |
| | TRAEEDEEEE |
| 51 | EAKEAEDGPM EESKPKPRSF MPNLVPPKIP DGERVDFDDI |
| | HRKRMEKDLN |
| 101 | ELQALIEAHF ENRKKEEEEL VSLKDRIERR RAERAEQQRI |
| | RNEREKERQN |
| 151 | RLAEERARRE EEENRRKAED EARKKKALSN MMHFGGYIQK |
| | QAQTERKSGK |
| 201 | RQTEREKKKK ILAERRKVLA IDHLNEDQLR EKAKELWQSI |
| | YNLEAEKFDL |
| 251 | QEKFKQQKYE INVLRNRIND NQKVSKTRGK AKVTGRWK |

(amino acid sequence of cTnI):
SEQ ID NO: 6

| | |
|---|---|
| 1 | MADGSSDAAR EPRPAPAPIR RRSSNYRAYA TEPHAKKKSK |
| | ISASRKLQLK |
| 51 | TLLLQIAKQE LEREAEERRG EKGRALSTRC QPLELAGLGF |
| | AELQDLCRQL |
| 101 | HARVDKVDEE RYDIEAKVTK NITEIADLTQ KIFDLRGKFK |
| | RPTLRRVRIS |
| 151 | ADAMMQALLG ARAKESLDLR AHLKQVKKED TEKENREVGD |
| | WRKNIDALSG |
| 201 | MEGRKKKFES |

(amino acid sequence of TnC):
SEQ ID NO: 7

| | |
|---|---|
| 1 | MDDIYKAAVE QLTEEQKNEF KAAFDIFVLG AEDGCISTKE |
| | LGKVMRMLGQ |
| 51 | NPTPEELQEM IDEVDEDGSG TVDFDEFLVM MVRCMKDDSK |
| | GKSEEELSDL |
| 101 | FRMFDKNADG YIDLDELKIM LQATGETITE DDIEELMKDG |
| | DKNNDGRIDY |
| 151 | DEFLEFMKGV E |

(amino acid sequence of pre-pro-BNP):
SEQ ID NO: 8

| | |
|---|---|
| 1 | MDPQTAPSRA LLLLLFLHLA FLGGRSHPLG SPGSASDLET |
| | SGLQEQRNHL |
| 51 | QGKLSELQVE QTSLEPLQES PRPTGVWKSR EVATEGIRGH |
| | RKMVLYTLRA |
| 101 | PRSPKMVQGS GCFGRKMDRI SSSSGLGCKV LRRH |

(amino acid sequence of pro-BNP):
SEQ ID NO: 9

| | |
|---|---|
| 1 | HPLGSPGSAS DLETSGLQEQ RNHLQGKLSE LQVEQTSLEP |
| | LQESPRPTGV |
| 51 | WKSREVATEG IRGHRKMVLY TLRAPRSPKM VQGSGCFGRK |
| | MDRISSSSGL |
| 101 | GCKVLRRH |

(amino acid sequence of NT-pro-BNP):
SEQ ID NO: 10

| | |
|---|---|
| 1 | HPLGSPGSAS DLETSGLQEQ RNHLQGKLSE LQVEQTSLEP |
| | LQESPRPTGV |
| 51 | WKSREVATEG IRGHRKMVLY TLRAPR |

(amino acid sequence of BNP):
SEQ ID NO: 11

| | |
|---|---|
| 1 | SPKMVQGSGC FGRKMDRISS SSGLGCKVLR RH |

(amino acid sequence of pre-pro-ADM):
SEQ ID NO: 12

| | |
|---|---|
| 1 | MKLVSVALMY LGSLAFLGAD TARLDVASEF RKKWNKWALS |
| | RGKRELRMSS |
| 51 | SYPTGLADVK AGPAQTLIRP QDMKGASRSP EDSSPDAARI |
| | RVKRYRQSMN |
| 101 | NFQGLRSFGC RFGTCTVQKL AHQIYQFTDK DKDNVAPRSK |
| | ISPQGYGRRR |
| 151 | RRSLPEAGPG RTLVSSKPQA HGAPAPPSGS APHFL |

(amino acid sequence of pro-ADM):
SEQ ID NO: 13

| | |
|---|---|
| 1 | ARLDVASEFR KKWNKWALSR GKRELRMSSS YPTGLADVKA |
| | GPAQTLIRPQ |
| 51 | DMKGASRSPE DSSPDAARIR VKRYRQSMNN FQGLRSFGCR |
| | FGTCTVQKLA |
| 101 | HQIYQFTDKD KDNVAPRSKI SPQGYGRRRR RSLPEAGPGR |
| | TLVSSKPQAH |
| 151 | GAPAPPSGSA PHFL |

(amino acid sequence of MR-pro-ADM):
SEQ ID NO: 14

| | |
|---|---|
| 1 | ELRMSSSYPT GLADVKAGPA QTLIRPQDMK GASRSPEDSS |
| | PDAARIRV |

(amino acid sequence of pre-pro-ET-1):
SEQ ID NO: 15

| | |
|---|---|
| 1 | MDYLLMIFSL LFVACQGAPE TAVLGAELSA VGENGGEKPT |
| | PSPPWRLRRS |
| 51 | KRCSCSSLMD KECVYFCHLD IIWVNTPEHV VPYGLGSPRS |
| | KRALENLLPT |
| 101 | KATDRENRCQ CASQKDKKCW NFCQAGKELR AEDIMEKDWN |
| | NHKKGKDCSK |
| 151 | LGKKCIYQQL VRGRKIRRSS EEHLRQTRSE TMRNSVKSSF |
| | HDPKLKGKPS |
| 201 | RERYVTHNRA HW |

(amino acid sequence of pro-ET-1):
SEQ ID NO: 16

| | |
|---|---|
| 1 | APETAVLGAE LSAVGENGGE KPTPSPPWRL RRSKRCSCSS |
| | LMDKECVYFC |
| 51 | HLDIIWVNTP EHVVPYGLGS PRSKRALENL LPTKATDREN |
| | RCQCASQKDK |
| 101 | KCWNFCQAGK ELRAEDIMEK DWNNHKKGKD CSKLGKKCIY |
| | QQLVRGRKIR |
| 151 | RSSEEHLRQT RSETMRNSVK SSFHDPKLKG KPSRERYVTH |
| | NRAHW |

(amino acid sequence of ET-1):
SEQ ID NO: 17

| | |
|---|---|
| 1 | CSCSSLMDKE CVYFCHLDII W |

(amino acid sequence of CT-pro-ET-1):
SEQ ID NO: 18

| | |
|---|---|
| 1 | RSSEEHLRQT RSETMRNSVK SSFHDPKLKG KPSRERYVTH |
| | NRAHW |

(amino acid sequence of Big-ET-1):
SEQ ID NO: 19

| | |
|---|---|
| 1 | CSCSSLMDKE CVYFCHLDII WVNTPEHVVP YGLGSPRS |

(amino acid sequence of PCT):
SEQ ID NO: 20

| | |
|---|---|
| 1 | APFRSALESS PADPATLSED EARLLLAALV QDYVQMKASE |
| | LEQEQEREGS |
| 51 | SLDSPRSKRC GNLSTCMLGT YTQDFNKFHT FPQTAIGVGA |
| | PGKKRDMSSD |
| 101 | LERDHRPHVS MPQNAN |

(amino acid sequence of pre-pro-ANP
(Homo sapiens)):
SEQ ID NO: 21

| | |
|---|---|
| 1 | MSSFSTTTVS FLLLLAFQLL GQTRANPMYN AVSNADLMDF |
| | KNLLDHLEEK |
| 51 | MPLEDEVVPP QVLSEPNEEA GAALSPLPEV PPWTGEVSPA |
| | QRDGGALGRG |
| 101 | PWDSSDRSAL LKSKLRALLT APRSLRRSSC FGGRMDRIGA |
| | QSGLGCNSFR |
| 151 | YRR |

(amino acid sequence of pro-ANP (Homo
sapiens)):
SEQ ID NO: 22

| | |
|---|---|
| 1 | NPMYNAVSNA DLMDFKNLLD HLEEKMPLED EVVPPQVLSE |
| | PNEEAGAALS |
| 51 | PLPEVPPWTG EVSPAQRDGG ALGRGPWDSS DRSALLKSKL |
| | RALLTAPRSL |
| 101 | RRSSCFGGRM DRIGAQSGLG CNSFRY |

(amino acid sequence of NT-proANP):
SEQ ID NO: 23

| | |
|---|---|
| 1 | NPMYNAVSNA DLMDFKNLLD HLEEKMPLED EVVPPQVLSE |
| | PNEEAGAALS |
| 51 | PLPEVPPWTG EVSPAQRDGG ALGRGPWDSS DRSALLKSKL |
| | RALLTAPR |

(MR-proANP = amino acid sequence of amino
acids 53-90 of proANP):
SEQ ID NO: 24

| | |
|---|---|
| 1 | PEVPPWT GEVSPAQRDG GALGRGPWDS SDRSALLKSK |
| | L |

(Myoglobin (Homo sapiens))
SEQ ID NO: 25

| | |
|---|---|
| 1 | MGLSDGEWQL VLNVWGKVEA DIPGHGQEVL IRLFKGHPET |
| | LEKFDKFKHL |
| 51 | KSEDEMKASE DLKKHGATVL TALGGILKKK GHHEAEIKPL |
| | AQSHATKHKI |
| 101 | PVKYLEFISE CIIQVLQSKH PGDFGADAQG AMNKALELFR |
| | KDMASNYKEL |
| 151 | GFQG |

(Creatine kinase (Homo sapiens))
SEQ ID NO: 26

| | |
|---|---|
| 1 | MPFSNSHNAL KLRFPAEDEF PDLSAHNNHM AKVLTPELYA |
| | ELRAKSTPSG |
| 51 | FTLDDVIQTG VDNPGHPYIM TVGCVAGDEE SYEVFKDLFD |
| | PIIEDRHGGY |
| 101 | KPSDEHKTDL NPDNLQGGDD LDPNYVLSSR VRTGRSIRGF |
| | CLPPHCSRGE |
| 151 | RRAIEKLAVE ALSSLDGDLA GRYYALKSMT EAEQQQLIDD |
| | HFLFDKPVSP |
| 201 | LLLASGMARD WPDARGIWHN DNKTFLVWVN EEDHLRVISM |
| | QKGGNMKEVF |
| 251 | TRFCTGLTQI ETLFKSKDYE FMWNPHLGYI LTCPSNLGTG |
| | LRAGVHIKLP |
| 301 | NLGKHEKFSE VLKRLRLQKR GTGGVDTAAV GGVFDVSNAD |
| | RLGFSEVELV |
| 351 | QMVVDGVKLL IEMEQRLEQG QAIDDLMPAQ K |

(C reactive Protein (Homo sapiens))
SEQ ID NO: 27

| | |
|---|---|
| 1 | MEKLLCFLVL TSLSHAFGQT DMSRKAFVFP KESDTSYVSL |
| | KAPLTKPLKA |
| 51 | FTVCLHFYTE LSSTRGYSIF SYATKRQDNE ILIFWSKDIG |
| | YSFTVGGSEI |
| 101 | LFEVPEVTVA PVHICTSWES ASGIVEFWVD GKPRVRKSLK |
| | KGYTVGAEAS |
| 151 | IILGQEQDSF GGNFEGSQSL VGDIGNVNMW DFVLSPDEIN |
| | TIYLGGPFSP |
| 201 | NVLNWRALKY EVQGEVFTKP QLWP |

1. A method for at least one of prognosis, risk assessment and diagnosis of a major adverse cardio- or cerebrovascular event (MACCE) in a patient who underwent gastrointestinal surgery, the method comprising the steps of:
i) providing a sample of a bodily fluid from said patient,
ii) determining in said sample the level of a biomarker selected from the group consisting of copeptin, troponin and brain natriuretic peptide (BNP),
iii) determining at least one additional parameter of said patient,
iv) combining the biomarker level determined in step ii) and the additional parameter determined in step iii) into a combined assessment, and
v) correlating the combined assessment to said at least one of prognosis, risk assessment and diagnosis of a MACCE in said patient.

2. The method according to claim 1,
wherein the method is a method for at least one of prognosis, risk assessment and diagnosis of a major adverse cardio- or cerebrovascular event (MACCE) in combination with an infection in a patient who underwent gastrointestinal surgery, preferably a method for risk stratification of said patients, stratifying said patients into a group more likely to develop both MACCE and an infection and another group less likely to develop both MACCE and an infection.

3. The method according to claim 1,
wherein the additional parameter is selected from the group consisting of the level of at least one additional biomarker and a clinical parameter of said patient.

4. The method according to claim 3,
wherein the additional biomarker is selected from the group consisting of copeptin, troponin, BNP, proadrenomedullin (proADM), preferably its fragment midregional proadrenomedullin (MR-proADM), proendothelin-1 (proET-1), preferably its fragment C-terminal proendothelin-1 (CT-proET-1), procalci-tonin (PCT), MR-proANP (mid-regional pro atrial natriuretic peptide), creatinine kinase, creatine kinase-MB, myoglobin, lactate and CRP (C-reactive protein).

5. The method according to claim 4,
wherein the levels of the biomarkers copeptin, troponin and brain natriuretic peptide (BNP) and, optionally, at least one of proADM, MR-proADM, proET-1, CTproET-1 and PCT are determined and combined into the combined assessment.

6. The method according to claim 3,
wherein the clinical parameter is selected from the group consisting of age, abnormal ECG, especially pathological Q-waves, LBBB, ST-elevation, ST-depression, T-wave inversion, intraoperative hypotension, intraoperative tachycardia, intraoperative bradycardia, hyperlipidaemia, smoking status, anaemia, functional capacities (METs), red-blood cell transfusion, arrhythmias, rhythm other than sinus, duration of the gastrointestinal surgery and operation size, patient history and liver disease.

7. The method according to claim 1,
wherein the level of at least one of the biomarkers is determined by determining the level of at least one of a precursor, a precursor fragment and a fragment of the biomarker.

8. The method according to claim 1,
wherein determining the level of troponin comprises determining the level of the subunit cardiac tro-ponin T (cTnT), preferably isoform 6 of cTnT or a homologous peptide with at least 75% amino acid sequence identity with isoform 6 of cTnT.

9. The method according to claim 1,
wherein determining the level of BNP comprises determining the level of precursor fragment NT-proBNP.

10. The method according to claim 1,
wherein the patient is at least 50 years old.

11. The method according to claim 1,
wherein the patient is treated with analgesics or pain treatment.

12. The method according to claim 1,
wherein the patient spent at least one day in a hospital after undergoing the gastrointestinal surgery.

13. The method according to claim 1,
wherein the gastrointestinal surgery is one of laparoscopic and open and is selected from the group consisting of
stomach surgery,
small intestine surgery, in particular duodenum surgery,
large intestine surgery, in particular rectum surgery,
reproductive system surgery, in particular hysterectomy or salpingoophorectomy,
kidney surgery, in particular nephrectomy,
urinary bladder surgery, in particular cystectomy,
gallbladder surgery, in particular cholecystectomy, and
surgery of gastrointestinal cysts.

14. The method according to claim 1,
wherein the patient is free from cardiovascular comorbidities.

15. The method according to claim 1,
wherein the patient is excluded if an exclusion condition is present in the patient, the exclusion condition being selected from the group consisting of organ transplantation, traumatic injury, endocrine disease, endocrine surgery, vascular disease, vascular surgery, endovascular disease, endovascular surgery, acute coronary syndrome (ACS), heart failure, decompensated congestive heart failure, aortic stenosis, reduced left ventricular ejection fraction (LVEF), and circulatory shock.

16. The method according to claim 1,
wherein the sample has been taken from the patient no more than 24 hours before the gastrointestinal surgery, no more than 24 hours after the gastrointestinal surgery, on day one after the gastrointestinal surgery, on day two after the gastrointestinal surgery or on day three after the gastrointestinal surgery.

17. The method according to claim 1,
wherein the method comprises providing of from two to five samples from the patient, wherein the samples are taken at different times before and/or after the gastrointestinal surgery, and wherein steps ii), iv) and v) are practiced on all said samples.

18. The method according to claim 17,
wherein the different times said of from two to five samples are taken are selected from the group con-sisting of no more than 24 hours before the gastrointestinal surgery, no more than 24 hours after the gastrointestinal surgery, day one after the gastrointestinal surgery, day two after the gastrointestinal surgery and day three after the gastrointestinal surgery.

19. The method according to claim 17,
wherein the levels of said biomarkers in the samples taken at different times are determined and then compared, and wherein the differences in the levels of the biomarkers at different times are combined into the combined assessment.

20. The method according to claim 1,
wherein a biomarker level determined to be above a specific threshold value is indicative of a MACCE in the patient.

21. The method according to claim 20,
wherein the specific threshold value is selected from the group consisting of
25 pmol/L, preferably 75 pmol/L, more preferably 125 pmol/L, even more preferably 175 pmol/L and most preferably 225 pmol/L for copeptin,
15 ng/L, preferably 20 ng/L, more preferably 25 ng/L, even more preferably 30 ng/L and most preferably 35 ng/L, 45 ng/L or 65 ng/L for cTnT,
500 ng/L, preferably 900 ng/L, more preferably 1300 ng/L, even more preferably 1700 ng/L and most preferably 2100 ng/L or 2800 ng/L for NT-proBNP,
0.8 nmol/L or 1.0 nmol/L, preferably 1.25 nmol/L, more preferably 1.5 nmol/L, even more preferably 1.75 nmol/L and most preferably 2.0 nmol/L or 2.4 nmol/L for MR-proADM,
80 pmol/L, preferably 90 pmol/L, more preferably 100 pmol/L, even more preferably 110 pmol/L and most preferably 120 pmol/L for CT-proET-1, and
0.5 μg/L, preferably 1.0 μg/L, more preferably 1.5 μg/L, even more preferably 2.0 μgL and most preferably 2.5 μg/L or 3.0 μg/L for PCT.

22. The method according to claim 1,
wherein the method comprises correlating the combined assessment to the risk of a MACCE in the patient within at least one of 30 days and 12 months after the gastrointestinal surgery.

23. The method according to claim 18,
wherein the prognosis comprises the risk of mortality within at least one of 30 days and 12 months after the gastrointestinal surgery.

24. The method according to claim 1,
wherein the MACCE is a myocardial injury after noncardiac surgery (MINS) and the method is a method for the diagnosis of a MINS in a patient who underwent gastrointestinal surgery.

25. The method according to claim 21,
wherein a biomarker level determined to be above a specific threshold value is indicative of a MINS in the patient.

26. The method according to claim 23,
wherein the specific threshold value is selected from the group consisting of
25 pmol/L, preferably 50 pmol/L or 75 pmol/L, more preferably 125 pmol/L, even more preferably 175 pmol/L and most preferably 225 pmol/L for copeptin,
10 ng/L, preferably 20 ng/L, more preferably 30 ng/L, even more preferably 40 ng/L and most preferably 50 ng/L or 60 ng/L for cTnT,
250 ng/L or 500 ng/L, preferably 750 ng/L, more preferably 1250 ng/L or 1500 ng/L, even more preferably 1750 ng/L and most preferably 2250 ng/L for NT-proBNP,
0.8 nmol/L or 1.0 nmol/L, preferably 1.25 nmol/L, more preferably 1.5 nmoVL, even more prefer-ably 1.75 nmol/L and most preferably 2.0 nmol/L for MR-proADM,
65 pmol/L or 75 pmol/L or 80 pmol/L, preferably 90 pmol/L, more preferably 100 pmol/L, even more preferably 110 pmol/L and most preferably 120 pmol/L for CT-proET-1, and
0.2 μg/L or 0.5 μg/L, preferably 0.75 μg/L or 0.8 μg/L, more preferably 1.0 μg/L, even more preferably 1.25 μg/L and most preferably 1.5 μg/L for PCT.

27. The method according to claim 17,
wherein the determined biomarker levels taken at different times are evaluated using different threshold values.

28. The method according to claim 17,
wherein the determined biomarker levels taken at different times are differently weighted.

29. The method according to claim 17,
wherein a relative change in the biomarker level between two samples taken at different times is combined into the combined assessment.

30. The method according to claim 1,
wherein a ratio between the levels of at least two biomarkers in the sample is determined and said ratio is combined into the combined assessment, wherein the biomarkers for determining the ratio are preferably selected from copeptin/tro-ponin, copeptin/BNP and BNP/tro-ponin.

31. The method according to claim 1,
wherein step iv) comprises providing reference data for the determined biomarkers and at least one value selected from the group consisting of the determined level of at least one of the biomarkers,
the differences in the level of at least one of the biomarkers taken at different times,
the relative change in the levels of one of said biomarkers determined in two samples taken at different times
the ratio between the levels of at least two biomarkers either taken at the same time or taken at different times and
the relative change of the ratios of at least two biomarkers determined in at least two samples taken at different times
is compared to the reference data, wherein the difference of the value as determined and the reference data is computed, and wherein the computed difference is preferably expressed in the form of a score, especially as a numerical value.

32. The method according to claim 31,
wherein at least two different scores are determined and the combined assessment comprises a combined score determined from said at least two different scores indicative of the presence or absence of a MACCE in the patient.

33. The method according to claim 31,
wherein the reference data pertains to patients who underwent gastrointestinal surgery and who did and/or did not have a MACCE or MINS.

34. The method according to claim 2,
wherein the infection is selected from the group consisting of a fungal, a bacterial or a viral infection.

35. The method according to claim 2,
wherein the infection is selected from the group consisting of a blood infection, a respiratory tract infection, a urinary tract infection, a skin infection or an abdominal cavity infection.

36. The method according to claim 2,
wherein the infection is selected from the group consisting of a blood stream infection, sepsis, severe sepsis and/or septic shock.

37. Kit for carrying out the method according to any one of the preceding claims, comprising:
at least one detection reagent for determining the level of at least one of the biomarkers copeptin, troponin and brain natriuretic peptide (BNP) in a sample of a bodily fluid from a patient, and
reference data from patients who underwent gastrointestinal surgery, particularly reference levels, corresponding to at least one of copeptin, troponin and BNP levels, wherein said reference data is preferably stored on a computer-readable medium and/or employed in the form of computer-executable code configured for comparing the determined level of at least one of copeptin, troponin and BNP to said reference data.

38. The kit according to claim 37,
wherein the at least one detection reagent comprises reagents for determining the levels of the biomarkers copeptin, troponin and brain natriuretic peptide (BNP) and, optionally, at least one of MR-proADM, CT-proET-1 and PCT in a sample of a bodily fluid from said patient, and wherein the reference data comprises data corresponding to said biomarkers in patients who underwent gastrointestinal surgery.

39. Computer, comprising means for running computer-executable code, with the computer-executable code being configured to carry out steps iv) and/or v) of the method of claim 1.

40. Computer according to claim 39,
wherein the computer is connected to an assay system, the assay system being configured to carry out step ii).

41. Computer program product comprising a set of computer instructions stored on at least one computer-readable medium, said set of computer instructions further comprising instructions executable by one or more processors to carry out steps iv) and/or v) of the method of claim 1.
